(19) United States
(12) Patent Application Publication
(10) Pub. No.: US 2005/0221304 A1
(43) Pub. Date: Oct. 6, 2005

(54) Modified random primers for probe labeling

(76) Inventors: Charlie Xiang, Germantown, MD (US); Michael J Brownstein, Rockville, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/474,611

(22) PCT Filed: Apr. 11, 2002
(86) PCT No.: PCT/US02/11656

§ 371 Date: Oct. 9, 2003

Related U.S. Application Data
(60) Provisional application No. 60/283,423, filed on Apr. 11, 2001.

(51) Int. Cl. C12Q 1/68; C12P 19/34

(52) U.S. Cl. 435/6; 435/91.2

(57) ABSTRACT

Methods are provided for labeling nucleic acid molecules for use in hybridization reactions, and kits employing these methods. The level of labeling is increased by including one or more reactive modifications, such as amine-modifications, into the primers used to initiate synthesis of the nucleic acid molecule. For instance through random-primed reverse transcription. Also provided are modified random primers (such as amine-modified random primers) useful in these methods, labeling and hybridization kits comprising such primers, labeled nucleic acid molecules and mixtures of molecules, and methods for using them.

### REFERENCE TO RELATED APPLICATIONS

This application claims the benefit of U.S. Provisional Application No. 60/283,423, filed Apr. 11, 2001, which is incorporated herein by reference.

### FIELD

This disclosure relates to methods of labeling nucleic acid probes for the detection of nucleic acids molecules, for instance producing labeled probes for detecting hybridization signals, such as those from a microarray.

### BACKGROUND OF THE DISCLOSURE

Microarray technology involves depositing nucleic acids (the target) on a solid platform (e.g., a glass microscope slide or chip) in a set pattern, and hybridizing a solution of labeled, potentially complementary nucleic acids (the probe) to the nucleic acid targets. This technology has been successfully applied to the simultaneous analysis of expression of many thousands of genes and large-scale gene discovery, as well as to polymorphism screening and mapping of genomic DNA clones. Microarray technology permits quantitative gene expression analysis using RNA transcripts from known and unknown genes, as well as qualitative detection of, for instance, human pathogens and disease-related genes from DNA samples.

Most applications using DNA arrays involve preparation of fluorescent labeled cDNA from the mRNA of the studied organism. The cDNA probes are then allowed to hybridize with the DNA fragments printed on the array, and the resulting hybridization profile is then scanned by a confocal microscope and analyzed by the appropriate software.

Two probe labeling strategies for microarray studies have been developed. The most commonly used involves directly incorporating fluorescent nucleotides (such as Cy3-dUTP and Cy5-dUTP) into cDNA probes through reverse transcription primed by an oligo dT primer (Duggan, et al., Nat. Genet. Suppl. 21:10-14, 1999). The optimal ratio of dye-modified to unmodified nucleotide used is governed by two factors: 1) that modified bases cause a deterioration in the strength and specificity of binding of probes to their target DNAs, and 2) that as many modified bases as possible have to be incorporated into probes to give good fluorescent signals. In practice, this trade-off limits the efficiency of probe labeling, and a large amount of starting RNA is required to produce labeled probe for each hybridization.

The second currently available labeling method is indirect labeling, wherein the cDNA is synthesized in the presence of amine-modified nucleotides (e.g. aminoallyl dUTP), and the fluorescent dyes are subsequently coupled onto the cDNA molecules by reaction with these amine groups. The same factors that limit the efficiency of direct labeling limit the efficiency of the indirect labeling method. Because of these problems, even optimal labeling reactions require a large quantity of mRNA (2 μg or more) or total RNA (20 μg or more) to produce enough probe to give a good hybridization signal. So much staring material is required that certain samples (such as clinical biopsies and microdissected cells) cannot be studied.

Recently, expensive, time consuming, multi-step procedures for amplifying and then labeling probe have been reported. These permit one to study much smaller samples than could be studied with conventional probe labeling methods. They are not ideal for routine studies, however, and are not sensitive enough for single cell experiments.

Protocols and reagents for conventional probe labeling are available commercially, for instance from companies that provide fluorescent-labeled nucleotides and kits for performing such labeling reactions (e.g., Amersham's CyScribe™ First-Strand cDNA Labeling Kit). Molecular Probes has recently released a new product line (ARES™ DNA Labeling Kits), which provides methods and reagents for incorporating aminoallyl-dUTP during the reverse transcription reaction, followed by addition of a reactive fluorescent dye, to produce labeled cDNA for various uses.

However, the existing nucleic acid/probe labeling methods do not provide good quality and high level labeling using very small amounts of starting nucleic acid. Therefore, there exists a need for a simple method of labeling nucleic acids from very small starting samples.

### SUMMARY OF THE DISCLOSURE

New methods are disclosed for preparing labeled nucleotide probes, which overcome several disadvantages of existing methods.

This disclosure provides methods for labeling nucleic acid molecules suitable for hybridization reactions, where the starting material for the labeling reaction can be minimal. In some embodiments, the starting material is a small number of cells, for instance as few as one cell. Provided methods enable labeling nucleic molecules contained within extremely small samples, including fine needle aspirates, tumor biopsies, tissue scrapes, laser-captured cells, and so forth, and thus enable genomic analysis of these samples using microarray and other high-throughput systems. In some embodiments, the starting material is less than about 10 cells, less than about 100 cells, or less than about 1000 cells. In another embodiment, the starting material is about 10 cells. In yet another embodiment, the starting material is about one cell. In some embodiments, the amount of starting material contains as little as 1-2 μg of total RNA. In certain embodiments, particularly those comprising an amplification, the amount of starting material may contain as little as about 50 pg to about 100 pg of total RNA.

Also disclosed are methods wherein modified nucleic acids are included in random primers that are used to initiate polymerization of a probe molecule, thereby introducing the modified nucleic acids consistently at the 5′ end of each probe molecule (such as cDNAs or fragments thereof). These methods maximize incorporation of modified nucleic acids into the resulting probe, thereby providing enhanced signal intensity and sensitivity in reactions using the probe, compared to the currently used methods.

In certain embodiments, the random primers include nucleotides that are modified by amine groups (such as aminoallyl moieties). Coupling of a fluorescent dye to the amine group can be performed after synthesis of the cDNA probe by reverse transcription. This novel labeling procedure provides for detection sensitivity at least two-fold enhanced compared to standard methods, and requires significantly less RNA.

In other embodiments, the modified nucleotides comprise a detectable molecule, such as a fluorophore or hapten.

One embodiment is a method of producing a modified nucleic acid probe, which method includes contacting a nucleic acid template with a modified random primer under conditions sufficient to permit base-specific hybridization between the template and the primer, and polymerizing a nucleic acid molecule complementary to a nucleic acid sequence in the template, thereby incorporating at least one modified oligonucleotide primer into the complementary nucleic acid, to produce a modified nucleic acid probe. The modified random oligonucleotide primer may comprise, for instance, an amine-modified dNTP or a label-substituted dNTP.

In specific embodiments where the modified nucleotide in the random primer comprises an amine-modified dNTP, the method further comprises coupling the modified nucleic acid probe to a label molecule (such as a fluorophore or hapten) to form a labeled probe (also referred to as a label-probe conjugate).

Also provided are modified random primers for use in the disclosed methods. Specific examples of such primers are shown in SEQ ID NOs: 1-10, for instance specifically the primers referred to as P2 (SEQ ID NO: 1) or P4 (SEQ ID NO: 2).

In particular embodiments, the nucleic acid template comprises a mixture of nucleic acid molecules, for instance a mixture of RNA molecules such as a preparation of total RNA, polyA RNA, or mRNA.

In particular embodiments, polymerizing comprises polymerizing a cDNA, for instance in a reverse transcription reaction where the template is an RNA molecule (or mixture thereof).

Also provided are methods of producing a fluorescent hybridization probe, which includes contacting a template nucleic acid sample with a modified random primer comprising at least one aminoallyl dNTP residue (such as an aminoallyl dUTP), polymerizing a nucleic acid molecule complementary to a sequence in the template sample and incorporating one or more modified random primers into that complementary molecule, to produce a modified complementary nucleotide. This modified complementary nucleotide can be contacted with an amine-reactive fluorescent label molecule, thereby producing a fluorescent hybridization probe. In specific embodiments, the modified complementary nucleotide is contacted with an amine-reactive hapten, or other amine-reactive molecule or group. Also encompassed herein are hybridization probes produced using these methods.

In certain methods provided herein, aminoallyl dUTP (or another modified nucleotide) is included during a polymerizing step.

This disclosure also provides an improved method for random primer reverse transcription labeling of a nucleic acid hybridization probe. One provided improvement is the use of random primers modified with at least one amine-substituted dNTP or fluorescent-dye modified dNTP to prime (initiate) the reverse transcription reaction. Improved hybridization probes produced by such methods are also provided.

Also provided are probe-labeling methods in which the template molecule is an amplified nucleic acid template. In one embodiment, the amplified template is RNA.

In certain disclosed methods the amplified template binds a second primer under conditions sufficient to permit base-specific hybridization between the template and the second primer. The second primer can include a T3 promoter and a random 9-mer (T3N9; SEQ ID NO: 12) and the second primer can be used in at least one round of cDNA synthesis other than the first round.

This disclosure also provides kits for producing a labeled hybridization probe or for probing an array, which kits include at least a modified random primer.

The foregoing and other features and advantages will become more apparent from the following detailed description of several embodiments, which proceeds with reference to the accompanying figures.

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NOs: 1 through 10 are several modified random primers.

SEQ ID NO: 11 is an oligo dT₍₁₅₎-T7 primer.

SEQ ID NO: 12 is a primer including the T3 promoter and a random 9-mer (13N9).

SEQ ID NO: 13 is an oligo dT₍₂₀₎-T7 primer.

### BRIEF DESCRIPTION OF THE FIGURES

FIG. 1 shows the structures of amine modified nucleotides dC-C6NH₂ and dT-C6NH₂, used for synthesis of amine modified random primers P2 and P4 (see Table 1).

FIG. 2 is a schematic representation of an example of a method for labeling probe molecules using amine modified primers during reverse transcription of cDNA from mRNA.

FIGS. 3A and 3B are scatter plots comparing the expression levels of genes between the same (FIG. 3A) or different (FIG. 3B) starting amount of RNA sample labeled with Cy5 (X-axis) and Cy3 (Y-axis), using amine-modified random primers (P2). The log-transformed fluorescence intensity of each spot is shown. There was a strong correlation between the signals in the two channels when either the same amount (R²=0.9901) or different amounts (R2=0.9904) were labeled.

FIG. 4 is a schematic representation of two different methods to amplify RNA. The method shown on the left uses random hexamers and T7-oligo dT primers for the second and subsequent rounds of cDNA synthesis. The method shown on the right uses a T3N9 primer for every round of cDNA synthesis except the first.

FIG. 5 is a series of scatter plot analyses showing the reliability of a disclosed labeling method throughout multiple rounds of amplification of RNA amplified with the T3N9 primer in cDNA microarray studies. These plots show quantification of the log of the fluorescent signal intensity of (A) total RNA versus RNA from first round amplification, (B) RNA from first round amplification versus RNA from second round amplification, (C)RNA from second round amplification versus RNA from third round amplification, (D) RNA from third round amplification versus RNA from fourth round amplification, (E) RNA from first round amplification versus RNA from third round amplification using T3N9 primers, and (F) RNA from first round amplification versus RNA from third round amplification using random hexamers and Ti-oligo dT primers.

### DETAILED DESCRIPTION

I. Abbreviations

aa-dNTP: aminoallyl-deoxy-nucleoside triphosphate
aRNA: amplified RNA
asRNA: antisense RNA
CDs: coding sequences
cRNA: copy RNA
dN6: random hexamer
dNTP: deoxy-nucleoside triphosphate
dA-C6-NH₂: amino allyl modified adenine
dC-C6-NH₂: amino allyl modified cytosine
dG-C₆—NH₂: amino allyl modified guanine
dT-C6-NH₂: amino allyl modified thymine
FISH: fluorescent in situ hybridization
ORF: open reading frame
PCR: polymerase chain reaction
RT: reverse transcription (transcriptase)
SSII RT: Superscript II reverse transcriptase
T3N9: primer including the T3 promoter and a random 9-mer
   II. Terms

Unless otherwise noted, technical terms are used according to conventional usage. Definitions of common terms in molecular biology may be found in Benjamin Lewin, Genes V, published by Oxford University Press, 1994 (ISBN 019-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Amplification: An increase in the amount of (number of copies of) nucleic acid sequence, wherein the increased sequence is the same as or complementary to the existing nucleic acid template. An example of amplification is the polymerase chain reaction, in which a biological sample collected from a subject is contacted with a pair of oligonucleotide primers, under conditions that allow for the hybridization (annealing) of the primers to nucleic acid template in the sample. The primers are extended under suitable conditions (though nucleic acid polymerization). If additional copies of the nucleic acid are desired, the first copy is dissociated from the template, and additional copies of the primers (usually contained in the same reaction mixture) are annealed to the template, extended, and dissociated repeatedly to amplify the desired number of copies of the nucleic acid.

The products of amplification may be characterized by electrophoresis, restriction endonuclease cleavage patterns, hybridization, ligation, and/or nucleic acid sequencing, using standard techniques.

Other examples of in vitro amplification techniques include reverse-transcription PCR (RT-PCR), strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Antisense RNA (asRNA): A molecule of RNA complementary to a sense (encoding) nucleic acid molecule. Often, asRNA is constructed by transcribing antisense strand RNA from a cDNA molecule.

Array: An arrangement of molecules, particularly biological macromolecules (such as polypeptides or nucleic acids) in addressable locations on a substrate. The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized so as to require microscopic examination, or other magnification, for evaluation.

Within an array, each arrayed molecule is addressable, in that its location can be reliably and consistently determined within the at least two dimensions of the array surface. In ordered arrays the location of each molecule sample can be assigned to the sample at the time when it is spotted onto the array surface, and a key may be provided in order to correlate each location with the appropriate target Often, ordered arrays are arranged in a symmetrical grid pattern, but samples could be arranged in other patterns (e.g., in radially distributed lines, spiral lines, or ordered clusters). Addressable arrays are computer readable, in that a computer can be programmed to correlate a particular address on the array with information (such as hybridization or binding data, including for instance signal intensity). In some examples of computer readable formats, the individual “spots” on the array surface will be arranged regularly in a pattern (e.g., a Cartesian grid pattern) that can be correlated to address information by a computer.

The sample application “spot” on an array may assume many different shapes. Thus, though the term “spot” is used, it refers generally to a localized deposit of nucleic acid, and is not limited to a round or substantially round region. For instance, substantially square regions of mixture application can be used with arrays encompassed herein, as can be regions that are substantially rectangular (such as a slot blot-type application), or triangular, oval, or irregular. The shape of the array substrate itself is also immaterial, though it is usually substantially flat and may be rectangular or square in general shape.

Binding or stable binding: An oligonucleotide binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional and physical binding assays. Binding may be detected functionally by determining whether binding has an observable effect upon a biosynthetic process such as expression of a coding sequence, DNA replication, transcription, amplification and the like.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, one method that is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt.

The binding between an oligomer and its target nucleic acid is frequently characterized by the temperature (Tₘ) (under defined ionic strength and pH) at which 50% of the target sequence remains hybridized to a perfectly matched probe or complementary strand. A higher (Tₘ) means a stronger or more stable complex relative to a complex with a lower (Tₘ).

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA extracted from cells or other samples.

Complementarity and percentage complementarity: Molecules with complementary nucleic acids form a stable duplex or triplex when the strands bind, (hybridize, anneal), to each other by forming Watson-Crick, Hoogsteen or reverse Hoogsteen base pairs. Stable binding occurs when an oligonucleotide remains detectably bound to a target nucleic acid sequence under the required conditions.

Complementarity is the degree to which bases in one nucleic acid strand base pair with the bases in a second nucleic acid strand. Complementarity is conveniently described by percentage, i.e. the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed), Molecular Cloning. A Laboratory Manual, 2nd ed., vol. 1-3, Cold Spring Harbor Laboratory Press, Cold Spring Harbor, N.Y., 1989.

Coupling: As used herein, the term “coupling” refers to the chemical reaction of a nucleotide, such as a modified nucleotide, with a detectable molecule, such as a hapten or label (e.g., a fluorophore). By way of example, disclosed embodiments of coupling reactions may be reactions between a nucleophile (functional group) and an electrophile, i.e., an electron poor reactive group. The coupling reaction may be facilitated by using an activating moiety to activate the electrophile to nucleophilic coupling. The activating group also usually is a leaving group. The nucleophile can be on either the nucleotide or on the detectable molecule, so long as the pair of reactants (nucleotide and detectable molecule) are capable of reacting with each other. Many embodiments have the nucleophile provided by the nucleotide.

Examples of reactions that may occur between the nucleophile and the electron poor reactive group include (in no particular order), but are not limited to, a Grignard reaction, a Wittig reaction, a condensation (such as an aldol condensation), a Mitsunobu reaction, formation of a Schiff base, and so forth.

Representative examples of nucleophilic functional groups include amines (—NH₂), —NHR (where R is aliphatic, e.g., an alkyl group), alcohols (—OH), thiols (—SH), acido-acetates, alkyl lithium components, and so forth. Hydrogen-bearing compounds also can be deprotonated to facilitate the coupling reaction. Additional examples of functional groups will be apparent to one of ordinary skill in the art.

Representative examples of leaving groups include halides (including F, Cl, and I), sulfonates, phosphates, DCC, EDC, imidazole, DMAP, DMF/acid chloride, and so forth. Further leaving groups are listed, for instance, in U.S. Pat. No. 5,268,486, and include isothiocyanate, isocyanate, monochlorotriazine, dichlortriazine, mono- or di-halogen substituted pyridine, mono- or di-halogen substituted diazine, maleimide, aziridine, sulfonyl halide, acid halide, hydroxysuccinimide ester, hydroxysulfosuccinimide ester, imido ester, hydrazine, azidonitrophenyl, azide, 3-(2-pridyl dithio)proprionamide, glyoxal and aldehyde. Additional examples of leaving groups will be apparent to one of ordinary skill in the art.

Specific examples of coupling reactions between aminoallyl nucleotides and fluorophores and haptens are illustrated in Nimmakayalu et al. (BioTechniques 28:518-522, 2000). Further specific examples are presented herein.

Fluorophore: A chemical compound, which when excited by exposure to a particular wavelength of light, emits light (i.e., fluoresces), for example at a different wavelength than that to which it was exposed. Fluorophores can be described in terms of their emission profile, or “color.” Green fluorophores, for example Cy3, FITC, and Oregon Green, are characterized by their emission at wavelengths generally in the range of 515-540 λ. Red fluorophores, for example Texas Red, Cy5 and tetramethylrhodamine, are characterized by their emission at wavelengths generally in the range of 590-690 λ.

Encompassed by the term “fluorophore” as it is used herein are luminescent molecules, which are chemical compounds which do not require exposure to a particular wavelength of light to fluoresce; luminescent compounds naturally fluoresce. Therefore, the use of luminescent signals eliminates the need for an external source of electromagnetic radiation, such as a laser. An example of a luminescent molecule includes, but is not limited to, aequorin (Tsien, 1998, Ann. Rev. Biochem. 67:509).

Examples of fluorophores are provided in U.S. Pat. No. 5,866,366. These include: 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron .RTM. Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other fluorophores include thiol-reactive europium chelates that emit at approximately 617 nm (Heyduk and Heyduk, Analyt. Biochem. 248:216-227, 1997; J. Biol. Chem. 274:3315-3322, 1999).

Other fluorophores include cyanine, merocyanine, styryl, and oxonyl compounds, such as those disclosed in U.S. Pat. Nos. 5,268,486; 5,486,616; 5,627,027; 5,569,587; and 5,569,766, and in published PCT patent application no. US98/00475, each of which is incorporated herein by reference. Specific examples of fluorophores disclosed in one or more of these patent documents include Cy3 and Cy5, for instance.

Other fluorophores include GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al., herein incorporated by reference) and derivatives thereof. Other fluorophores are known to those skilled in the art, for example those available from Molecular Probes (Eugene, Oreg.).

Particularly useful fluorophores have the ability to be attached to (coupled with) a nucleotide, such as a modified nucleotide, are substantially stable against photobleaching, and have high quantum efficiency.

High throughput genomics: Application of genomic or genetic data or analysis techniques that use microarrays or other genomic technologies to rapidly identify large numbers of genes or proteins, or distinguish their structure, expression or function from normal or abnormal cells or tissues.

Human Cells: Cells obtained from a member of the species Homo sapiens. The cells can be obtained from any source, for example peripheral blood, urine, saliva, tissue biopsy, surgical specimen, amniocentesis samples and autopsy material. From these cells, genomic DNA, cDNA, mRNA, RNA, and/or protein can be isolated.

Hybridization: Oligonucleotides (and oligonucleotide analogs) hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning. A Laboratory Manual, 2nd ed., vol. 1-3, Cold Spring Harbor Laboratory Press, Cold Spring Harbor, N.Y., 1989, chapters 9 and 11, herein incorporated by reference.

For purposes of the present invention, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in an oligonucleotide/polynucleotide. A nucleotide sequence refers to the sequence of bases in an oligonucleotide/polynucleotide.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Inosine is also a base that can be integrated into DNA or RNA in a nucleotide (dITP or ITP, respectively).

Modified nucleotide (modified nucleoside triphosphate): A modified nucleotide is a nucleotide that has been modified, for example a nucleotide to which a chemical moiety has been added, usually one that gives an additional functionality to the modified nucleotide. Generally, the modification comprises a functional group or a leaving group, and permits coupling of the nucleotide to a detectable molecule, such as a fluorophore or hapten. In other embodiments, an alteration in the structure of the nucleotide or a deletion of an atom can make the nucleotide reactive with a detectable label.

For instance, one specific class of modifications are those that add a reactive amine group to the nucleotide; an aminoallyl group is one such amine modification. Amine groups are reactive with a wide spectrum of other chemical groups, which will be known to one of ordinary skill in the art. By way of example, amine groups are reactive with intermediate N-hydroxysuccinimide (NHS) esters, such as those on NHS ester cyanine dyes. Amine groups also can be reacted with peptide molecules (such as antigenic fragments or antibody or antibody fragment) or biotin (for instance, to which a fluorescent dye can then be coupled), for instance. Examples of amine-reactive fluorophores that can be coupled to amine modified-nucleotides include, but are not limited to, fluorescein, BODIPY, rhodamine, Texas Red, cyanine dyes, and their derivatives. Reaction of amine-reactive fluorophores usually proceeds at pH values in the range of pH 7-10.

Alternatively, thiol-reactive fluorophores can be used to generate a fluorescently-labeled nucleotide or oligonucleotide. Thus, also contemplated herein are nucleotides (and oligonucleotides) containing a thiol group as its modification. Reaction of fluors with thiols usually proceeds rapidly at or below room temperature (RT) in the physiological pH range (pH 6.5-8.0) to yield chemically stable thioesters. Examples of thiol-reactive fluorophores include, but are not limited to: fluorescein, BODIPY, cumarin, rhodamine, Texas Red and their derivatives.

Other functional groups that can be added to a nucleotide to make a modified nucleotide include alcohols and carboxylic acids. These reactive functional groups also can be used to couple a fluorophore to the nucleotide or oligonucleotide.

In particular embodiments, fluorescently-labeled nucleotides/oligonucleotides have a high fluorescence yield, and retain the critical features of the nucleotide/oligonucleotide, primarily the ability to bind to a complementary strand of a nucleic acid molecule and prime a polymerizing reaction.

The term also include nucleotides containing modified bases, modified sugar moieties and modified phosphate backbones, for example as described in U.S. Pat. No. 5,866,336 to Nazarenko et al. (herein incorporated by reference).

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Also included in the term “modified nucleotide” are branched nucleotides bearing more than one modification. Examples of branched nucleotides are disclosed, for instance, in Horn and Urdea (Nuc. Acids Res. 17:6959-6967, 1989) and Nelson et al. (Nuc. Acids Res. 17:7179-7186, 1989), incorporated herein by reference. The inclusion of branched modified nucleotides in modified random primers disclosed herein can provide even higher levels of labeling, since each branched modified nucleotide can accept more than one detectable molecule in a coupling reaction (or series of such reactions), one at each modification.

Specific examples of modified nucleotides, and oligonucleotides comprising such modified nucleotides, are provided in U.S. Pat. Nos. 4,605,735; 4,667,025; and 4,489,336, for instance, which patents are incorporated herein by reference.

In certain embodiments, modifications to nucleotides allow for incorporation of the nucleotide into a growing nucleic acid chain, for instance through in vitro chemical synthesis (e.g., by phosphoramidite synthesis).

Oligonucleotide: An oligonucleotide is a plurality of nucleotides joined by phosphodiester bonds, between about 6 and about 300 nucleotides in length. An oligonucleotide analog refers to compounds that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

A modified oligonucleotide (or modified nucleic acid molecule) is one that comprises at least one modified nucleotide. Modified oligonucleotides may be mono-modified (i.e., carrying only one modified nucleotide) or poly-modified (carrying more than one modified nucleotide, either more than one of a single type or one or more each of multiple types). The primer described herein as “P2” is an example of a mono-modified oligonucleotide. The primer described herein as “P4” is an example of a poly-modified oligonucleotide.

Particular oligonucleotides and modified oligonucleotide can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 8-25 bases, such as 8, 12, 15 or 20 bases.

Peptide Nucleic Acid (PNA): An oligonucleotide analog with a backbone comprised of monomers coupled by amide (peptide) bonds, such as amino acid monomers joined by peptide bonds.

Polymerization: Synthesis of a new nucleic acid chain (oligonucleotide or polynucleotide) by adding nucleotides to the hydroxyl group at the 3′-end of a pre-existing RNA or DNA primer using a pre-existing DNA strand as the template. Polymerization usually is mediated by an enzyme such as a DNA or RNA polymerase. Specific examples of polymerases include the large proteolytic fragment of the DNA polymerase I of the bacterium E. coli (usually referred to as Kleenex polymerase), E. coli DNA polymerase I, and bacteriophage T7 DNA polymerase. Polymerization of a DNA strand complementary to an RNA template (e.g., a cDNA complementary to a mRNA) can be carried out using reverse transcriptase (in a reverse transcription reaction).

For in vitro polymerization reactions, it is necessary to provide to the assay mixture an amount of required cofactors such as M⁺⁺, and dATP, dCTP, dGTP, dTTP, ATP, CTP, GTP, UTP or other nucleoside triphosphates, in sufficient quantity to support the degree of amplification desired. The amounts of deoxyribonucleotide triphosphates substrates required for polymerizing reactions are well known to those of ordinary skill in the art. Nucleoside triphosphate analogues or modified nucleoside triphosphates can be substituted or added to those specified above.

Primer: Primers are relatively short nucleic acid molecules, usually DNA oligonucleotides six nucleotides or more in length. Primers can be annealed to a complementary target DNA strand (“priming”) by nucleic acid hybridization to form a hybrid between the primer and the target DNA strand, and then the primer extended along the target DNA strand by a nucleic acid polymerase enzyme. Pairs of primers can be used for amplification of a nucleic acid sequence, e.g. by nucleic-acid amplification methods known in to those of ordinary skill in the art.

A primer is usually single stranded, which may increase the efficiency of its annealing to a template and subsequent polymerization. However, primers also may be double stranded. A double stranded primer can be treated to separate the two strands, for instance before being used to prime a polymerization reaction (see for example, Nucleic Acid Hybridization. A Practical Approach. Hames and Higgins, eds., IRL Press, Washington, 1985). By way of example, a double stranded primer can be heated to about 90°-100° C. for about 1 to 10 minutes.

Probe: A probe comprises an isolated nucleic acid attached to a detectable label or other reporter molecule, or a mixture of such nucleic acids; also referred to as a labeled probe or labeled primer. Typical labels include radioactive isotopes, enzyme substrates, co-factors, ligands, chemiluminescent or fluorescent agents, haptens, and enzymes. A modified probe is a probe that contains at least one modified nucleotide residue, e.g. at least one aminoallyl-dUTP for instance.

Probe standard: A probe molecule for use as a control in analyzing an array. Positive probe standards include any probes that are known to interact with at least one of the nucleic acids of the array, which may be found in certain spots, or in all spots on the array, each spot containing a mixture (e.g., a different mixture) of nucleic acid molecules. Negative probe standards include any probes known not to interact with any nucleic acid sequence contained in at least one mixture of nucleic acids of the array.

Such a control probe sequence could, for instance, be designed to hybridize with a so-called “housekeeping” gene, which is known to or suspected of maintaining a relatively constant expression level (or at least known to be expressed) in a plurality of cells, tissues, or conditions. Many of such “housekeeping” genes are well known; specific examples include histones, β-actin, or ribosomal subunits (either mRNA encoding for ribosomal proteins or rRNAs). Housekeeping genes can be specific for the cell type being assayed, or the species or Kingdom from which sample nucleic acid mixtures have been produced. For instance, ribulose bis-phosphate carboxylase oxygenase (RuBisCO), an enzyme involved in plant metabolism, may provide useful positive control probes for use with arrays if the nucleic acid mixtures arrayed have been derived from plant cells or tissues. Likewise, probes from the RuBisCO sequence (or any other plant-specific sequence) could provide good negative controls for gene profiling array spots that include animal-derived samples.

In some instances, as in certain of the kits that are provided herein, a probe standard will be supplied that is unlabeled. Such unlabeled probe standards can be used in a labeling reaction as a standard for comparing labeling efficiency of the test probe that is being studied. In some embodiments, labeled probe standards will be provided in the kits.

Probing: As used herein, the term “probing” refers to incubating an array with a probe molecule (usually in solution) in order to determine whether the probe molecule will hybridize to molecules immobilized on the array. Synonyms include “interrogating,” “challenging,” “screening” and “assaying” an array. Thus, an array is said to be “probed” or “assayed” or “challenged” when it is incubated with (hybridized to) a probe molecule.

Purified: The term purified does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid may be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid content of the preparation.

Random Primer: An primer with a random sequence (see, for instance, U.S. Pat. Nos. 5,043,272 and 5,106,727, incorporated herein by reference).

“Random” sequence means that the positions of alignment and binding (annealing) of the primers to a template nucleic acid molecule are substantially indeterminate with respect to the template under conditions wherein the primers are used to initiate polymerization of a complementary nucleic acid. Methods for estimating the frequency at which an oligonucleotide of a certain sequence will appear in a nucleic acid polymer are described in Volinia et al. (Comp. App. Biosci. 5: 33-40, 1989).

The sequences of random primers may not be random in the absolute mathematic sense. For instance, chemically synthesized random primers will be random to the extent that physical and chemical efficiencies of the synthetic procedure will allow, and based on the method of synthesis. Random primers derived from natural sources (e.g., through digestion of an existing polynucleotide) may be less random, due to favored arrangements of bases in the source organism. Oligonucleotides having defined sequences may satisfy the definition of random if the conditions of their use cause the locations of their apposition to the template to be indeterminate. Also, random primers may be “random” only over a portion of their length, in that one residue within the primer sequence, or a portion of the sequence, can be identified and defined prior to synthesis of the primer. Thus, any primer type is defined to be random so long as the positions along the template nucleic acid strand at which primed nucleic acid extension occurs is largely indeterminate.

Random primers may be generated using available oligonucleotide synthesis procedures; randomness of the sequence may be introduced by providing a mixture of nucleic acid residues in the reaction mixture at one or more addition steps (to produce a mixture of oligonucleotides with random sequence). Thus, a random primer can be generated by sequentially incorporating nucleic acid residues from a mixture of 25% of each of dATP, dCTP, dGTP, and dTTP, to form an oligonucleotide. Other ratios of dNTPs can be used (e.g. more or less of any one dNTP, with the other proportions adapted so the whole amount is 100%).

The term “random primer” specifically includes a collection of individual oligonucleotides of different sequences, for instance which can be indicated by the generic formula 5′-XXXXX-3′, wherein X represents a nucleotide residue that was added to the oligonucleotide from a mixture of a definable percentage of each dNTP. For instance, if the mixture contained 25% each of dATP, dCTP, dGTP, and dTTP, the indicated primer would contain a mixture of oligonucleotides that have a roughly 25% average chance of having A, C, G, or T at each position.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g. by genetic engineering techniques.

RNA: A typically linear polymer of ribonucleic acid monomers, linked by phosphodiester bonds. Naturally occurring RNA molecules fall into three general classes, messenger (mRNA, which encodes proteins), ribosomal (rRNA, components of ribosomes), and transfer (tRNA, molecules responsible for transferring amino acid monomers to the ribosome during protein synthesis). Messenger RNA includes heteronuclear (hnRNA) and membrane-associated polysomal RNA (attached to the rough endoplasmic reticulum). Total RNA refers to a heterogeneous mixture of all types of RNA molecules.

Sample: Includes biological samples such as those derived from a human or other animal source (for example, blood, stool, sera, urine, saliva, tears, biopsy samples, histology tissue samples, cellular smears, moles, warts, etc.); bacterial or viral preparations; cell cultures; forensic samples; agricultural products; waste or drinking water; milk or other processed foodstuff; air; and so forth. Samples containing a small number of cells can be acquired by any one of a number of methods, such as fine needle aspiration, micro-dissection, biopsy, tissue scrapes, or laser capture micro-dissection. Samples can also be diluted to a level where they contain as few as 100 cells, 10 cells or even as few as 1 cell in a sample.

Stripping: Bound probe molecules can be stripped from an array, for instance a cDNA array, in order to use the same array for another probe interaction analysis (e.g., to determine gene expression level in a different cell sample). Any process that will remove substantially all of the prior probe molecule from the array, without also significantly removing the immobilized nucleic acid targets of the array, can be used. By way of example only, one method for stripping an array is by boiling it in stripping buffer (e.g., very low or no salt with 0.1% SDS), for instance for about an hour or more. The stripped array may be washed, for instance in an equilibrating or low stringency buffer, prior to incubation with another probe molecule.

Where a stripability enhancer (such as the nucleotide analog of the StripAble™ and Strip-EZ™ system from Ambion (Austin, Tex.)) is used, the procedures provided by the manufacturer for use with this product provide a good starting point for tailoring probing and stripping conditions for use with arrays. Addition of stripability enhancers to probes for use with arrays is optional.

Subject: Living, multicellular vertebrate organisms, a category that includes both human and veterinary subjects for example, mammals, birds and primates.

Template: A nucleic acid polymer that can serve as a substrate for the synthesis of a complementary nucleic acid strand. Nucleic acid templates may be in a double-stranded or single-stranded form. If the nucleic acid is double-stranded at the start of the polymerization reaction, it may be treated to denature the two strands into a single-stranded, or partially single-stranded, form. Methods are known to render double-stranded nucleic acids into single-stranded, or partially single-stranded, forms, such as by heating to about 90°-100° C. for about 1 to 10 minutes, or by alkali treatment, such as at a pH of 12 or greater.

A template nucleic acid molecule may be either DNA or RNA and may be either homologous to the source or heterologous to the source of the sample in which it is contained, or both. For example, amplification of a template in human tissue sample infected with a virus may result in amplification of both viral and human sequences.

Nucleic acid synthesis (polymerization) in a “template dependent manner” refers to synthesis wherein the sequence of the newly synthesized strand of nucleic acid is essentially dictated by complementary base pairing to the sequence of a template nucleic acid strand.

In some embodiments, a template nucleic acid may be amplified prior to using it to produce a nucleic acid probe using the modified random primers provided herein. For instance, an amplified template can be produced by amplifying (through one or more rounds of amplification) mRNA molecules. Examples of methods for amplifying mRNAs are described in Examples 4 and 5. In certain embodiments, it is beneficial if the amplification of the template molecule is such that the amplified template reflects the relative abundance of the sequences found in the original template molecules. See also Wang et al. (Nature Biotech 18:457459, 2000), co-assigned U.S. provisional patent application No. 60/192,700, filed Mar. 28, 2000, and the related PCT application (No. US01/09993), filed Mar. 28, 2001, each of which is incorporated herein by reference.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “of” is intended to include “and” unless the context clearly indicates otherwise. Hence “comprising A or B” means including A, or B, or A and B. “Comprising” means “including.” It is further to be understood that all base sizes or amino acid sizes, and all molecular weight or molecular mass values, given for nucleic acids or polypeptides, are approximate and are provided for descriptive purposes. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Modified Random Primers

DNA microarray technology has become one of the most important tools for high throughput studies in medical research, with applications in the areas of gene discovery, gene expression, and genetic mapping. Much progress has been made for making high quality microarrays through improving the surface materials and fabrication techniques, but little has been achieved for the labeling methods to increase the detection signal and sensitivity which limits the application of DNA microarray technology in certain areas including clinical diagnosis. Gene expression studies and clinical diagnosis using tissue biopsies or small cell populations have in the past often been difficult due to the limited availability of RNA, because prior methods of labeling cDNA probes for microarray hybridization require substantial amounts of RNA to generate the probes.

Prior labeling techniques involve incorporating fluorescent dye conjugated nucleotides such as Cy3-/Cy5-dUTP/dCTP, or other modified nucleotides like aminoallyl dUTP (aa-dUTP), during probe polymerization (e.g., during reverse transcription of cDNA from mRNA). The optimal ratio of dye-modified to unmodified nucleotide used is governed by two factors: 1) that modified bases cause a deterioration in the strength and specificity of binding of probes to their target DNAs, and 2) that as many modified bases as possible have to be incorporated into probes to give good fluorescent signals. In practice, this trade-off limits the efficiency of probe labeling, and a large amount of starting RNA is required to produce labeled probe for each hybridization.

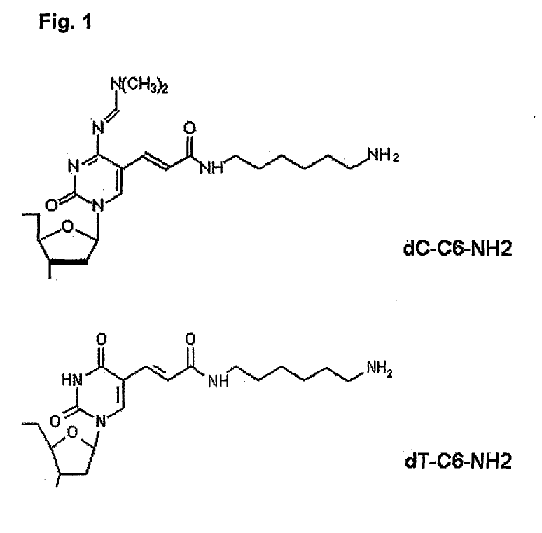

This disclosure provides methods for producing modified (e.g., labeled) nucleic acid molecules useful as probes, for instance for hybridization to microarrays, which overcome disadvantages of prior labeling methods. The probes provided herein have at least one label at their 5′ end and they are more highly labeled than those produced using previous methods. The improved labeling is acheived through the incorporation of one or more chemically modified nucleotides (such as those shown in FIG. 1) into random primers, which are then used to initiate synthesis (polymerization) of the probe. These methods enable the efficient production of probe nucleic acid incorporating the modification throughout the length of the molecule, without substantially decreasing label incorporation or efficiency of the polymerization reaction.

Because nucleic acid probes produced using these methods are intensely labeled, less probe is needed in order to be reliably detected, for instance in a hybridization reaction. As illustrated in the Examples and accompanying figures, hybridization probes produced using mono-modified primer embodiments (such as the primer referred to herein as P2) consistently provide reliable hybridization signals. Thus, the herein-described labeling methods can be used to reliably label very small amounts of starting material for analysis, such as expression analysis using microarrays.

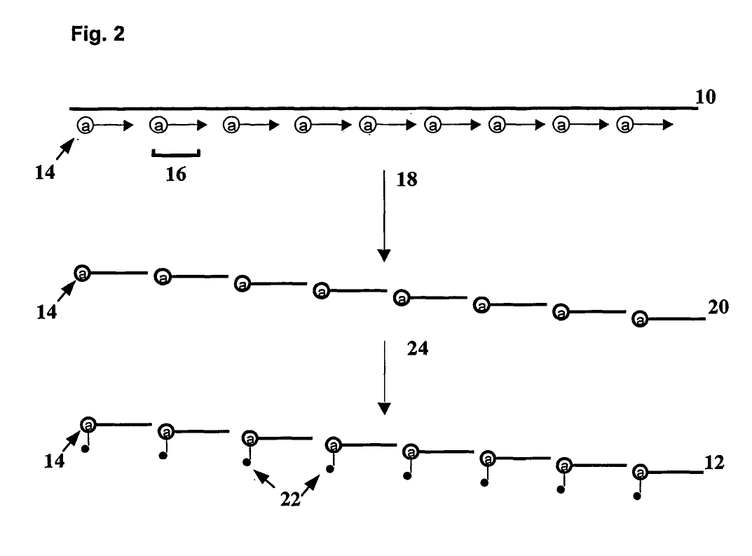

One specific encompassed method is shown schematically in FIG. 2. In this illustrated method, mRNA (10) is used as the template to produce modified (in this case, fluorescently labeled) cDNA fragments (12). A modified nucleotide (14) (such as the amine-modified nucleotide aminoallyl dUTP) is incorporated directly into random primers (16) that are then used to prime reverse transcription (18) of the mRNA, producing amine-modified cDNA fragments (20). These fragments may be, but need not be, full length cDNAs. After synthesis, label moieties (22) can be added to the modified cDNA fragments (20) at the modification groups (14) (e.g., the amine groups of the amine-modified nucleotides) to produce labeled cDNA probe (12). This probe (12) in certain embodiments will be a mixture of labeled cDNA molecules, some of which will be fragments of what would be considered “full-length” cDNAs.

Because modified nucleotides are incorporated directly into the random primers, these methods result in reliable incorporation of a high level of reactive groups (and labels) into each probe molecule from a small amount of starting total RNA, without substantially inhibiting the RT reaction. In some circumstances this provides a stronger fluorescent signal, and may provide more consistent and reproducible fluorescent signal, compared to standard RT methods using unlabeled random primers in the presence of modified individual nucleic acids. Thus, an effective probe can be produced, and clear signals read from a microarray, even when using significantly less starting nucleic acid (as little as about 1-2 μg of total RNA). This enables microarray analysis of gene expression from much smaller samples. This labeling protocol is very simple and considerably less expensive than methods currently considered to be state of the art.

In certain embodiments, particularly those comprising an amplification process, the amount of starting material (e.g., a preparation of nucleic acids, a lysed cell sample, etc.) may contain less than about 1 μg of total RNA. In other embodiments, the amount of starting material may contain less than about 2 μg of total RNA, less than about 3 μg of total RNA, less than about 5 μg of total RNA, or less than about 10 μg of total RNA.

In other embodiments, the starting material comprises as few as about 1 cell, about 10 cells, about 100 cells, about 200 cells, about 500 cells, about 750 cells, or about 1000 cells.

In one specific example, random hexamers and T7-oligo dT primers are used for the second and subsequent rounds of RNA amplification. By way of example, primers including the T3 promoter and a random ninemer (T3N9, SEQ D NO: 12) can be used for the second and subsequent rounds of RNA amplification, thereby incorporating the T3 RNA polymerase promoter sequence into the nucleic acid at random locations based on the random ninemer (9-mer). Other promoters could be used.

In some embodiments, additional amine-modified dUTP (or another dNTP) optionally can be included in the RT reaction, thereby incorporating additional amine-reactive groups into the cDNA during synthesis. This method can increase the labeling intensity and therefore is suitable in certain circumstances.

Random primers have been widely used in labeling the DNA probes with radioisotope from DNA template (Feinberg and Vogelstein, Anal. Biochem. 132:6-13, 1983; Swensen, BioTechniques, 20:486-491, 1996). They have also been used in priming the cDNA synthesis from purified mRNA (Lear et al., BioTechniques 18:78-83, 1995; Allawi et al., RNA 7:314-327, 2001). Because the largest portion in total RNA pool is ribosomal RNA, using total RNA as template material to generate cDNA probes may increase non-specific hybridization from microarrays. However, under highly stringent condition for hybridization and washing steps, represented by the conditions presented herein, such problems have been avoided.

Choice of Modification

Many examples of modified nucleotides are provided herein. The choice of which modification to use on a random primer provided herein will be influenced by the specific use to which the labeled probe is to be put, and the detectable molecule to be coupled to the nucleotide. For instance, the detectable molecule must be able to couple with the modified nucleotide; one should comprise a nucleophilic reactive group, while the other contains an electron poor reactive center and a leaving group.

FIG. 1 and Table 1 show structures of specific examples of modified nucleotides and specific amine modified random primers (P2 and P4) made with these nucleotides. The amine-modified nucleotides are incorporated into the oligonucleotide primers during regular DNA chemical synthesis. Amine modified dT and dC are commercially available in a form that can be used for DNA synthesis, for instance from Sigma (St Louis, Mo.), or from Glen Research in Virginia Additional modified nucleotides, and sources, are listed in Example 6.

It is contemplated that other modified nucleotides are also useful in the described methods. For instance, nucleotides that carry a label or other detectable molecule are considered to be modified, and can be used to generate the modified primers employed in methods described herein. Methods for making such labeled nucleotides, and examples thereof, are described in further detail in Example 6.

Synthesis of Oligonucleotide Primers

In vitro methods for the synthesis of oligonucleotides are well known to those of ordinary skill in the art; such conventional methods can be used to produce primers for the disclosed methods. The most common method for in vitro oligonucleotide synthesis is the phosphoramidite method, formulated by Letsinger and further developed by Caruthers (Caruthers et al., Chemical synthesis of deoxyoligonucleotides, in Methods Enzymol. 154:287-313, 1987). This is a non-aqueous, solid phase reaction carried out in a stepwise manner, wherein a single nucleotide (or modified nucleotide) is added to a growing oligonucleotide. The individual nucleotides are added in the form of reactive 3′-phosphoramidite derivatives. See also, Gait (Ed.), Oligonucleotide Synthesis. A practical approach, IRL Press, 1984.

In general, the synthesis reactions proceed as follows: First, a dimethoxytrityl or equivalent protecting group at the 5′ end of the growing oligonucleotide chain is removed by acid treatment. (The growing chain is anchored by its 3′ end to a solid support such as a silicon bead.) The newly liberated 5′ end of the oligonucleotide chain is coupled to the 3′-phosphoramidite derivative of the next deoxynucleoside to be added to the chain, using the coupling agent tetrazole. The coupling reaction usually proceeds at an efficiency of approximately 99%; any remaining unreacted 5′ ends are capped by acetylation so as to block extension in subsequent couplings. Finally, the phosphite triester group produced by the coupling step is oxidized to the phosphotriester, yielding a chain that has been lengthened by one nucleotide residue. This process is repeated, adding one residue per cycle. See, for instances, U.S. Pat. Nos. 4,415,732, 4,458,066, 4,500,707, 4,973,679, and 5,132,418. Oligonucleotide synthesizers that employ this or similar methods are available commercially (e.g., the PolyPlex oligonucleotide synthesizer from Gene Machines, San Carlos, Calif.). In addition, many companies will perform such synthesis (e.g., Sigma-Genosys, TX; Operon Technologies, CA; Integrated DNA Technologies, IA; and TriLink BioTechnologies, CA).

Modified nucleotides, such as aminoallyl dNTPs or dNTPs carrying a fluorescent dye (such as Cy3 or Cy5), can be incorporated into an oligonucleotide essentially as described above for non-modified nucleotides.

Random primers may be generated using known chemical synthesis procedures; randomness of the sequence may be introduced by providing a mixture of nucleic acid residues in the reaction mixture at one or more addition steps (to produce a mixture of oligonucleotides with random sequence). See, for instance, U.S. Pat. Nos. 5,043,272 and 5,106,727. A random primer preparation (which is a mixture of different oligonucleotides, each of determinate sequence) can be generated by sequentially incorporating nucleic acid residues from a mixture of, for instance, 25% of each of dATP, dCTP, dGTP, and dTTP, (or a modified dNTP such as aa-dUTP). Other ratios of dNTPs can be used (e.g., more or less of any one dNTP, with the other proportions adapted so the whole amount is 100%). Likewise, in the synthesis of a random primer, the synthesizer can be programmed to introduce one or more known residues (such as one or more specific nucleotide residues or modified nucleotide residues) at a defined location within the primer. For instance, a defined sequence can be included at the 5′ or 3′ end of the primer, or in the middle of the primer (with random sequences to the 5′ and 3′ end), or a combination of these.

By way of further example, the following modified random primers are contemplated:

TABLE 1
Amine Modified Random Primers

| | | |
|---|---|---|
| | | SEQ |
| Primer | Sequence | ID NO: |

| | | |
|---|---|---|
| P2 | [AC6T]--NNNNNN * | 1 |
| P4 | XXXXXN ** | 2 |
| Pr A | [AC6T]--INNNNNN *** | 3 |
| Pr B | [AC6T]--[AC6T]--INNNNNN *** | 4 |
| Pr C | [AC6T]--I--[AC6T]--INNNNNN *** | 5 |
| Pr D | [AC6T]--II--[AC6T]--INNNNNN *** | 6 |
| Pr E | [AC6T]--III--[AC6T]--INNNNNN *** | 7 |
| Pr F | [AC6T]--IIII--[AC6T]--INNNNNN *** | 8 |
| Pr G | [AC6T]--IIIIII--[AC6T]--INNNNNN *** | 9 |
| Pr H | X-NNNNNN **** | 10 |

* [AC6T]: 100% T-C₆-NH₂; N: 25% each of G, C, A, T.
** 25% G, 25% A, 25% C-C₆-NH₂, 25% T-C₆NH₂; N: 25% each of G, C, A, T.
*** I = inosine; N: 25% each of G, C, A, T.
**** X = 50% T-C6-NH₂, 50% C-C6-NH₂, or X = 25% T-C6-NH_{2+L, 25% C-C6-NH}₂, 25% G-C6-NH₂and 25% A-C6-NH₂; N: 25% each of G, C, A, T.

Choice of Detectable Molecule

Though most of the examples presented herein refer to the addition of a fluorescent label (particularly Cy3 or Cy5) to the modified nucleotide that is incorporated in a primer used in the described methods, other detectable molecules are contemplated.

DNA molecules containing a primary amino group (e.g., attached to the C6 or C2 carbon) can be coupled with a standard peptide or can interact with any intermediate N-hydroxysuccinimide (NHS) ester. In an embodiment disclosed herein, amine modified dT and dC nucleotides are added in place of thymidine and cytidine residues during oligonucleotide synthesis. After deprotection of the modified group, the primary amine on (for instance) the C6 moiety is spatially separated from the oligonucleotide by a spacer arm with a total of 10 atoms, and can be reacted with a label molecule or attached to an enzyme or any other reactive peptide or protein. Thus, for instance, the provided methods for making amine modified DNA can be used to produce modified probe molecules that comprise a peptide antigen or single chain antibody, which can be used in detection assays involving antigen and antibody reactions.

Thus, in particular embodiments, the provided primers are linked to a hapten such as biotin, or a fluorescent dye. For instance, any NHS-ester dyes can be used in DNA probe labeling with the provided amine modified random primers.

In addition, it is contemplated that in those embodiments in which the modification of the nucleotide is a label (e.g., a fluorescent dye molecule) or other detectable molecule, the modified primer is the labeled primer and can be used to produce labeled probe without requiring a subsequent chemical modification.

Applications

Because the disclosed labeling methods require very little starting material, even as little as one cell, these methods open up conventional cDNA microarray analysis to entire new fields of research, particularly those in which the source material was heretofore too scarce to permit cDNA array analysis (e.g., for samples acquired by fine needle aspirates or micro-dissection, or experimental models studying embryonic tissue or small organisms). For instance, these methods can be used to study specific cell populations within the brain or from embryonic cell samples (e.g. to study embryonic development). Gene expression within individual white blood cells, such as those from peripheral blood cells, or other potentially unique cells, can be assessed using these methods. Within a tissue biopsy, different cell populations can be sampled (e.g. through laser capture microdissection) and the expression levels of genes in the different cell populations assayed.

Similarly to regular random primers, the provided amine modified random primers also can be used in many applications such as RT-PCR, FISH and others in which fluorescent dyes are utilized for signal detection. For instance, the provided modified primer labeling system can be used to make labeled probes from DNA templates using E. coli DNA polymerase I by random priming labeling.

Previous Methods of Labeling cDNA Probes

For the sake of comparison, the following is a representative example of prior methods for labeling hybridization cDNA probes for use in microarray analysis. This method produces fluorescently labeled cDNA using traditional primers (oligo(dT) or unmodified random primers) and reverse transcription PCR. The presented method is adapted from those available at the Internet site of the National Human Genome Research Institute, National Institutes of Health, Bethesda, Md.

Using an anchored oligo dT primer, the primer is annealed to the RNA in the following 17 μl reaction (use a 0.2 ml PCR tube so that incubations can be carried out in a PCR cycler):

| Component | for Cy5 labeling | | for Cy3 labeling | |
|---|---|---|---|---|
| Total RNA (>7 mg/ml) | 150-200 | μg | 50-80 | μg |
| Anchored primer (2 μg/μl) | 1 | μl | 1 | μl |
| DEPC H₂O | to 17 | μl | to 17 | μl |

If using an oligo dT(12-18) primer, the primer is annealed to the RNA in the following 17 μl reaction:

| Component | for Cy5 labeling | | for Cy3 labeling | |
|---|---|---|---|---|
| Total RNA (>7 mg/ml) | 150-200 | μg | 50-80 | μg |
| dT(12-18) primer (1 μg/μl) | 1 | μl | 1 | μl |
| DEPC H₂O | to 17 | μl | to 17 | μl |

The incorporation rate for Cy5-dUTP is less than that of Cy3-dUTP, so more RNA is labeled to achieve more equivalent signal from each labeled species.

The samples are then heated to 65° C. for 10 minutes and cooled on ice for 2 minutes. To each tube, add 23 μl of reaction mixture (below) containing either Cy5-dUTP or Cy3-dUTP nucleotide, mix well by pipetting and use a brief centrifuge spin to concentrate the reaction in the bottom of the tube.

| Reaction Mixture | μl |
|---|---|
| 5x first strand buffer | 8 |
| 10x low T dNTPs mix | 4 |
| Cy5 dUTP or Cy3 dUTP (1 mM) | 4 |
| 0.1 M DTT | 4 |
| RNasin (30 u/μl) | 1 |
| Superscript II (200 u/μl) | 2 |
| Total volume | 23 |

Superscript polymerase is sensitive to denaturation at air/liquid interfaces, so care is exercised to suppress foaming in handling of this reaction.

The polymerization reaction is incubated at 42° C. for 30 minutes. An additional 2 μl of Superscript II is added, well mixed into the reaction volume, and incubated at 42° C. for an additional 30-60 minutes.

To stop the reaction, 5 μl of 0.5M EDTA is added, followed by 10 μl 1N NaOH. The sample is incubated at 65° C. for 30-60 minutes to hydrolyze residual RNA, then cooled to room temperature. The reaction must be stopped by addition of EDTA before the NaOH is added, since nucleic acids precipitate in alkaline magnesium solutions. Also, the purity of the sodium hydroxide solution is important; slight contamination or long term storage in a glass vessel can produce a solution that will degrade the Cy5 dye, turning the solution yellow.

The reaction is neutralized by adding 25 μl of 1M Tris-HCl (pH 7.5). The labeled cDNA is desalted using a MicroCon 100 cartridge. The neutralized reaction, 400 μl of TE pH 7.5 and 20 μg of human C0t-1 DNA are added to the cartridge and mixed by pipetting. The column is spun for 10 minutes at 500×g, then washed by adding 200 μl TE pH 7.5. The sample is concentrated to about 20-30 μl by spinning at 500×g for approximately 8-10 minutes.

Alternatively, a smaller pore MicroCon 30 cartridge can be used to speed the concentration step. In this case, centrifuge the first wash is performed for approximately 4.5 minutes at 16,000×g and the second (200 μl wash) for about 2.5 minutes at 16,000×g.

The neutralized and desalted sample is recovered by inverting the concentrator cartridge over a clean collection tube and spinning for three minutes at 500×g.

In some cases, the Cy5-labeled cDNA will form a gelatinous blue precipitate that is recovered in the concentrated volume. This indicates that the sample was contaminated. The more extreme the contamination, the greater the fraction of cDNA the will be captured in this gel. Even if heat solubilized, this material tends to produce uniform, non-specific binding to the DNA targets.

When concentrating by centrifugal filtration, the times required to achieve the desired final volume are variable. Overly long spins can remove nearly all the water from the solution being filtered. When fluor-tagged nucleic acids are concentrated on the filter in this fashion, they are very hard to remove from the cartridge. It is beneficial to approach the desired volume by conservative approximations of the required spin times. If control of volumes proves difficult, the final concentration can be achieved by evaporating liquid in the speed-vacuum. Vacuum evaporation, if not to complete dryness, does not degrade the performance of the labeled cDNA.

A 2-3 μl aliquot of the Cy5 labeled cDNA probe can be used for quality analysis (leaving 18-28 μl for hybridization). Run this probe on a 2% agarose gel (for instance, 6 cm wide×8.5 cm long, 2 mm wide teeth) in Tris Acetate Electrophoresis Buffer (TAE). For maximal sensitivity when running samples on a gel for fluor analysis, loading buffer with minimal dye is used, and ethidium bromide is not added to the gel or running buffer.

The resultant gel can be scanned on a Molecular Dynamics Storm fluorescence scanner (setting: red fluorescence, 200 micron resolution, 1000 volts on PMT). Successful labeling produces a dense smear of probe from 400 bp to >1000 bp, with little pile-up of low molecular weight transcripts. Weak labeling and significant levels of low molecular weight material indicate a poor labeling reaction. A fraction of the observed low molecular weight material is unincorporated fluor nucleotide, and should be expected in any reaction.

Computer Assisted (Automated) Detection and Analysis of Array Hybridization

The data generated by assaying an array can be analyzed using known computerized systems. For instance, the array can be read by a computerized “reader” or scanner and quantification of the binding of probe to individual addresses on the array carried out using computer algorithms. Likewise, where a control probe (such as a probe prepared from a control cell or sample with known expression levels) has been used, computer algorithms can be used to normalize the hybridization signals in the different spots of the array. Such analyses of an array can be referred to as “automated detection” in that the data is being gathered by an automated reader system.

In the case of labels that emit detectable electromagnetic wave or particles, the emitted light (e.g., fluorescence or luminescence) or radioactivity can be detected by very sensitive cameras, confocal scanners, image analysis devices, radioactive film or a Phosphoimager, which capture the signals (such as a color image) from the array. A computer with image analysis software detects this image, and analyzes the intensity of the signal for each probe location in the array. Signals can be compared between spots on a single array, or between arrays (such as a single array that is sequentially probed with multiple different probe molecules), or between the labels of different probes on a single array.

Computer algorithms can also be used for comparison between spots on a single array or on multiple arrays. In addition, the data from an array can be stored in a computer readable form.

Certain examples of automated array readers (scanners) will be controlled by a computer and software programmed to direct the individual components of the reader (e.g., mechanical components such as motors, analysis components such as signal interpretation and background subtraction). Optionally software may also be provided to control a graphic user interface and one or more systems for sorting, categorizing, storing, analyzing, or otherwise processing the data output of the reader.

To “read” an array, an array that has been assayed with a detectable probe to produce binding (e.g., a binding pattern) can be placed into (or onto, or below, etc., depending on the location of the detector system) the reader and a detectable signal indicative of probe binding (hybridization) detected by the reader. Those addresses at which the probe has bound to an immobilized nucleic acid on the array provide a detectable signal, e.g. in the form of electromagnetic radiation. These detectable signals could be associated with an address identifier signal, identifying the site of the “positive” hybridized spot. The reader gathers information from the addresses, associates it with the address identifier signal, and recognizes addresses with a detectable signal as distinct from those not producing such a signal. Certain readers are also capable of detecting intermediate levels of signal, between no signal at all and a high signal, such that quantification of signals at individual addresses is enabled.

Certain readers that can be used to collect data from the arrays, especially those that have been probed using a fluorescently tagged probe, will include a light source for optical radiation emission. The wavelength of the excitation light will usually be in the UV or visible range, but in some situations may be extended into the infra-red range. A beam splitter can direct the reader-emitted excitation beam into the object lens, which for instance may be mounted such that it can move in the x, y and z directions in relation to the surface of the array substrate. The objective lens focuses the excitation light onto the array, and more particularly onto the (polypeptide) targets on the array. Light at longer wavelengths than the excitation light is emitted from addresses on the array that contain fluorescently-labeled probe molecules (i.e., those addresses containing a nucleic acid molecule within a spot containing a nucleic acid molecule to which the probe binds).

In certain embodiments, the array may be movably disposed within the reader as it is being read, such that the array itself moves (for instance, rotates) while the reader detects information from each address. Alternatively, the array may be stationary within the reader while the reader detection system moves across or above or around the array to detect information from the addresses of the array. Specific movable-format array readers are known and described, for instance in U.S. Pat. No. 5,922,617, hereby incorporated in its entirety by reference. Examples of methods for generating optical data storage focusing and tracking signals are also known (see, for example, U.S. Pat. No. 5,461,599, hereby incorporated in its entirety by reference).

For the electronics and computer control, a detector (e.g., a photomultiplier tube, avalanche detector, Si diode, or other detector having a high quantum efficiency and low noise) converts the optical radiation into an electronic signal. An op-amp first amplifies the detected signal and then an analog-to-digital converter digitizes the signal into binary numbers, which are then collected by a computer.

The following examples are provided to illustrate certain particular features and/or embodiments. These examples should not be construed to limit the invention to the particular features or embodiments described.

### EXAMPLES

### Example 1

### Production of Primers for cDNA Labeling

Oligo (dT)12-18 primer (referred to herein as P0) was purchased from GIBCO BRL Life Technologies (Rockville, Md.); it is supplied in a pre-made solution at a concentration of 500 μg/ml.

Unmodified, random hexamer primer (referred to herein as P1) was purchased from Operon Technologies (New Orleans, La.) and was dissolved in DEPC treated H₂O at the concentration of 1 μg/μl.

Currently, amine-modified nucleotides dT and dC are available from Glen Research (Sterling, Va.). FIG. 1 shows the structures of these amine-modified nucleotides. Using these modified nucleotides, two different amine modified random primers (referred to herein as P2 and P4; Table 2) were synthesized using in vitro chemical synthesis using the phosphoramidite method (Caruthers et al., Chemical synthesis of deoxyoligonucleotides, in Methods Enzymol. 154:287-313, 1987). The oligonucleotides were dissolved in DEPC treated H₂O at the concentration of 1 μg/μl for use in reverse transcription reactions.

TABLE 2
Amine Modified Random Primers

| | | | |
|---|---|---|---|
| Primer | Sequence (5′ to 3′) | Manufacturer | SEQ ID NO: |

| | | | |
|---|---|---|---|
| P2 | [AC6T]NNNNNN * | SIGMA Genosys (The Woodland, TX) | 1 |
| P4 | XXXXXN ** | TriLink Bio Technologies (San Diego, | 2 |
| | | CA) | |

* [AC6T]: 100% T-C6-NH₂; N: 25% each of G, C, A, T.
** X 25% G, 25% A, 25% C-C6-NH₂, 25% T-C6-NH₂; N: 25% each of G, C, A, T.

### Example 2

### Generation of Fluorescent Probe

This example describes methods for producing labeled cDNA using amine modified primers P2 and P4 and reverse transcription in the presence of aminoallyl dUTP.

Template RNA:

RNAs from mouse C2 and NIH 3T3 cell lines were isolated using ThIzol reagent from GIBCO BRL Life Technologies (Rockville, Md.) followed by extraction with RNeasy kit from Qiagen (Valencia, Calif.). RNAs from mouse 18 day embryo and liver were also extracted with the combination of TRIzol reagent and RNeasy kit

Production of cDNA Probe

Primer (P0, P1, P2, or P4) was annealed to the RNA in the following manner:

Primer (2 μl), total RNA (0.1-5 μg in 15.5 μl), and RNase inhibitor (1 μl, Promega, Madison, Wis.) were mixed, and the RNA/primer mixture incubated at 70° C. for 10 minutes, then chilled on ice immediately for 10 minutes to encourage annealing of the primers.

A 17 μl aliquot of the RT mix (6 μl 5× first strand buffer (provided with SSII RT), 6 μl 5× aa-dUTP/dNTPs, 3 μl 0.1M DTT, and 2 μl SuperScript II Reverse transcriptase (SSII RT; GIBCO BRL Life Technologies, Rockville, Md.) was added to each primer-RNA mix, for a total volume of 30 μl, and the sample incubated at 42° C. for 2 hours to permit reverse transcription of the cDNA. aa-dUTP (5-[3-aminoallyl]-2′-deoxyuridine 5′-triphosphate) was from Sigma, St. Louis, Mo.

The reverse transcription reaction was stopped by the addition of 10 μl of 0.5M EDTA. RNA was degraded from the reaction mixture by adding 10 μl of 1N NaOH, and incubating the sample at 65° C. for 30 minutes. The reaction was neutralized by adding 10 μl of 1M HCl.

Various methods can be used to clean up the neutralized cDNA sample. In one method, the cDNA was cleaned up using a MinElute PCR purification kit. The microcentriflge tube was filled with 300 μl Buffer PB and 60 μl of the neutralized reaction solution, essentially as provided by the manufacturer, was added to the Buffer PB. The MinElute column was placed in a 2 ml collection tube in a suitable rack. To bind DNA, the sample was applied to the MinElute column and centrifuged for 1 minute. For optimal recovery, all traces of sample were transferred to the column. The flow-through was poured back into the column and centrifuged again for 1 minute. The flow-through was then discarded. The MinElute column was washed by placing it back into the original collection tube, adding 750 μl of Buffer PE then incubating it for 5 minutes at room temperature. The column was then centrifuged for 1 minute, the flow-through discarded and the MinElute column placed back in the same tube. The column was centrifuged for an additional 1 minute at maximum speed to remove residual buffer PE, then placed in a clean 1.5 ml microcentriflge tube.

To elute the DNA, 10 μl of water (pH between 7.0 and 8.5) was added to the center of the membrane within the column. After 1 minute the column was centrifuged for 5 minutes. The average eluate was 9 μl out of the 10 μl applied. DNA was eluted from the column twice more with 10 μl of water, collecting a total of 27 μl of purified cDNA.

In another method, the neutralized cDNA sample was cleaned up using a MicroCon 100 concentrator cartridge (Millipore, Bedford, Mass.). The cartridge was primed with 450 μl of ddH₂O, then the cartridge was spun at 13,000 rpm for about 3 minutes. The flow-through was discarded, and the cDNA on the cartridge was washed twice with 500 μl of ddH₂O. The cDNA sample was eluted from the cartridge, and dried in a speed vacuum.

Coupling,

Various coupling methods can be used. Monofunctional NHS-ester Cy3 and Cy5 dyes and the dNTPs used in the coupling were from Amersham Pharmacia (Piscataway, N.J.).

In one method, 3 μl of 1M sodium bicarbonate, pH 9.3 was added to the 27 μl of cDNA purified from the MinElute column, followed by the addition of 1 μl of dye solution (NHS-ester Cy3 or Cy5, 62.5 μg/μl in DMSO). The solution was mixed by pipetting up and down several times. The tubes were wrapped in aluminum foil, and incubated at room temperature for one hour in an orbital shaker (USA Scientific, Ocala, Fla.)).

In another method, a 5× aa-dUTP/dNTPs solution was made as follows: 10 μl each of dATP (100 mM), dGTP (100 mM), and dCTP (100 mM); 4 μl of aa-dUTP (100 mM), 6 μl of dTTP (100 mM) were combined with 360 μl of DEPC-treated H₂O. The monofunctional NHS-ester Cy3 and Cy5 dyes were first resuspended in 72 μl of dd H₂O and aliquots of 4.5 μl were placed in tubes. The aliquots were dried in a speed vacuum and stored in −20° C. freezer. The dried dyes were re-suspended in 4.5 μl of 100 mM sodium bicarbonate before mixing with cDNA made from reverse transcription reactions.

The cDNA sample dried in a speed vacuum was resuspended in 4.5 μl water. Pre-dried aliquots of monofunctional NHS-ester Cy3 or Cy5 dye were resuspended in 4.5 μl of 100 mM NaBicarbonate Buffer (pH 9.0). One aliquot of cDNA was mixed with one aliquot of Cy3 or Cy5, and the samples incubated at RT for 1 hour in dark to couple the fluorescent dye to the modified cDNA.

Quenching and Cleanup

The fluorescence was quenched by adding 4.5 μl of 4M hydroxylamine to the dye-coupled cDNA, and incubating the mixture at RT for 30 minutes in dark. The labeled sample was cleaned up using a Qiagen Qia-quick PCR purification kit (Qiagen, Valencia, Calif.) as follows:

The Cy3 and Cy5 labeled reactions were mixed, and 30 μl water and 500 μl Buffer PB (provided with the kit) added. This diluted sample was applied to the Qia-quick column, and the column spun at 13,000 rpm in for 1 minute. The flow-through was removed by aspiration, and the column washed twice with 750 μl Buffer PE (provided with the kit), spun out for 1 minute and the flow-through aspirated off each time. The column was then transferred to a fresh tube, and 30 μl of Buffer EB (provided with the kit) added to column. This was incubated for 1 minute at RT to elute the probe, then the column spun at 13,000 rpm for 1 minute and the eluate collect. The elution step was repeated, the sample combined with the first eluate, for a total collected volume of approximately 60 μl.

### Example 3

### Microarray Hybridization

This example provides a method for analyzing cDNA microarrays using labeled probe produced by the amine modified random primer method, such as that produced by the method of Example 2. The signal generated from microarray hybridization using cDNAs produced using amine-modified random primers is more consistent and more reliable than that obtained with previously known methods that use traditional random or oligo-dT primers.

Microarray

cDNA microarrays with 10,752 mouse clones were fabricated on glass slides using OmniGrid from GeneMachines (San Carlos, Calif.) using standard techniques.

Hybridization and Analysis

The labeled cDNA eluate produced in Example 2 was dried in a speed vacuum, and brought up in ddH₂O to a final volume of 23 μl. To this was added 4.5 μl of 20×SSC, 2 μl of poly A (10 mg/ml), and 0.6 μl of 10% SDS. The nucleic acids were denatured at 100° C. for 2 minutes, then applied to a prepared microarray. The microarray was permitted to hybridize by incubation for 16-24 hours in a 65° C. water bath.

After hybridization, the slide was washed at room temperature for 10 minutes each in the following solutions: (a) 0.5×SSC, 0.01% SDS, (b) 0.06×SSC. The washed slide was spun (in a tube or a slide rack) at 800 rpm at room temperature for five minutes to dry.

Microarray hybridization images were scanned with GenePix 4000A scanner from Axon (Foster City, Calif.), and the resultant data analyzed with IPLab (Fairfax, Va.) and ArraySuite (Chen, NHGR1). To determine the reliability of each ratio measurement, a set of quality indicators was used. An intensity measurement in either channel is determined to be unreliable if it fails to satisfy any one of the following conditions: 1) The number of pixels associated with the element must be sufficiently large. 2) The local background must be flat. 3) The signal consistency within the target area must be uniform. 4) The majority of the signal pixels should not be saturated. For each ratio measurement, red (R)/green (G), one further condition is imposed—the average signal, (R+G)/2, must be three times the noise level (Chen et al. Biomedical Optics. 2, 364-374, 1997).

### RESULTS AND DISCUSSION

P0 Versus P2, Using Half as Much Template RNA

Oligo dT primer (P0) and amine modified random primer (P2) were directly compared. 2.5 μg total RNA was used for labeling with random primer P2; twice that amount was used with oligo dT primer P0. The two labeled probes were then hybridized to each of two identical arrays on the same slide. The slide was scanned at same laser power and PMT level. The images were processed and analyzed with IPLab/ArraySuite. The hybridization color pattern with the amine modified random primer P2 was exactly same as the pattern with the oligo dT primer. While the amount of RNA used with the amine modified random primer P2 was only half that used with oligo dT primer P0, the observed hybridization intensities were similar to those obtained with the P2 primer.

The Pearson's correlation was calculated from the two ratio sets and scatter plots were generated; the calculated Pearson's r value was 0.8006 for the P0/P2 comparison. This was similar to that observed when two arrays both hybridized with probe prepared with primer P2 were compared (Pearson's r value of 0.8143).

P0 Versus P2, Using 10-Fold Less Template RNA

Microarray hybridization was compared using probes produced with amine modified random primer P2 (Y-axis) and direct labeling technique using oligo dT primer P0% X-axis). Five μg of either mouse NIH 3T3 or mouse C2 total RNA was used to produce labeled cDNA with amine 10 modified random primer P2; in contrast, 50 μg of mouse NIH 3T3 or C2 total RNA was used to obtain a readable signal using the traditional direct labeling protocol primed by the oligo(dT) primer P0. By using the amine modified random primer P2, it was demonstrated that only one tenth of starting material was needed to generate very similar hybridization signal intensities.

Differentially Expressed Genes (P0 Versus P2, Using 10-Fold Less Template RNA)

Table 3 includes a list of 95 genes that are differentially expressed in mouse 3T3 versus C2 cells (3T3/C2 ratios ≧3 or ≦1/3). The table shows the results of six array experiments. Three 9568-element arrays were interrogated with oligo-dT primed probes, and three others were interrogated with amine modified hexamer primed probes. Fifty fig of RNA were used for each oligo-dT primed labeling and 5 μg were used for each modified hexamer primed labeling. Array images were analyzed using ArraySuite software. Low quality ratios were filtered as described above.

When genes were searched that were 3-fold over- or under-expressed by the two cell lines studied, 99 genes were found with the oligo-dT priming method and 102 genes were found with the amine-modified method. Among these, 95 genes were the same. The ratios of the 95 genes that were differentially expressed are quite consistent among all six experiments. Elements representing the RhoB and four-and-a-half LIM domains 1 transcripts were printed two and three times on the array, respectively. These genes appear to be expressed at a higher level in C2 than 3T3 cells, and it is convincing that all elements representing them showed similar ratios. The four-and-a-half LIM domains protein is known to be produced in cardiac and skeletal muscle.

TABLE 3

| GenBank | | | | | | | | |
|---|---|---|---|---|---|---|---|---|
| No | Unigene No | dTa | dTb | dTc | P2a | P2b | P2c | Clone description |
| AI849214 | Mm.105330 | 18.3218 | 17.5265 | 16.2183 | 20.7580 | 25.5790 | 22.7692 | whey acidic protein |
| AI848293 | Mm.34507 | 6.9711 | 5.3423 | 3.5613 | 8.1450 | 6.4472 | 6.7642 | ESTs |
| AI847098 | Mm.29982 | 5.2438 | 4.5541 | 4.5709 | 5.6932 | 5.1469 | 5.7707 | ERO1-like (S. cerevisiae) |
| AI852317 | Mm.4063 | 3.7839 | 3.5895 | 4.4260 | 5.4368 | 5.0830 | 5.2313 | N-myc downstream |
| | | | | | | | | regulated 1 |
| AI844828 | Mm.2834 | 3.7150 | 3.7105 | 3.9967 | 5.1164 | 4.9883 | 4.7269 | glycine transporter 1 |
| AI846827 | Mm.70667 | 5.2250 | 4.0641 | 3.4577 | 4.6474 | 4.2576 | 4.2443 | Mus musculus, Similar to |
| | | | | | | | | oxidation resistance 1 |
| AI843085 | Mm.157648 | 5.5280 | 4.4538 | 4.7526 | 4.5177 | 4.0203 | 4.3967 | RIKEN cDNA |
| | | | | | | | | 5730403B10 gene |
| AI842716 | Mm.140158 | 5.5015 | 5.8588 | 5.1314 | 4.4732 | 4.4336 | 4.4295 | cytochrome P450, 51 |
| AI836864 | Mm.4704 | 6.6261 | 5.7066 | 3.7317 | 4.4534 | 3.9560 | 4.4178 | forkhead box G1 |
| AI853347 | Mm.21884 | 4.0523 | 3.9847 | 3.3449 | 4.4364 | 4.7968 | 4.3672 | ESTs, Weakly similar to |
| | | | | | | | | GTPase-activating protein |
| | | | | | | | | SPA-1 |
| AI843677 | Mm.45357 | 3.7376 | 3.5943 | 3.5423 | 3.8309 | 3.4541 | 3.3920 | Erbb2 interacting protein |
| AI838612 | Mm.14601 | 3.0926 | 3.3974 | 3.2623 | 3.6027 | 3.4499 | 3.4159 | glutathione S-transferase, |
| | | | | | | | | mu 2 |
| AI848205 | Mm.35844 | 3.6669 | 3.3875 | 3.1423 | 3.4911 | 3.0100 | 3.1019 | growth arrest specific 5 |
| AI850589 | Mm.22627 | 3.7784 | 3.1037 | 3.1616 | 3.2339 | 3.2407 | 3.6818 | epidermal growth factor |
| | | | | | | | | receptor pathway substrate |
| | | | | | | | | 15 |
| AI852765 | Mm.24193 | 0.3300 | 0.3343 | 0.3183 | 0.3254 | 0.2847 | 0.3249 | glypican 1 |
| AI836264 | Mm.4871 | 0.1492 | 0.1253 | 0.1183 | 0.3200 | 0.3100 | 0.2357 | tissue inhibitor of |
| | | | | | | | | metalloproteinase 3 |
| AI844851 | Mm.10406 | 0.3209 | 0.3235 | 0.2910 | 0.3243 | 0.2993 | 0.3025 | RIKEN cDNA |
| | | | | | | | | 3110001M13 gene |
| AI851985 | Mm.29586 | 0.2668 | 0.2559 | 0.2278 | 0.3233 | 0.2814 | 0.3107 | RIKEN cDNA |
| | | | | | | | | 2610024P12 gene |
| AI845475 | Mm.30811 | 0.1031 | 0.1333 | 0.1210 | 0.3180 | 0.3200 | 0.3100 | ESTs |
| AI853172 | Mm.27173 | 0.2968 | 0.3133 | 0.3032 | 0.3132 | 0.2847 | 0.3100 | ectoplacental cone |
| | | | | | | | | sequence |
| AI835858 | Mm.27685 | 0.2834 | 0.2925 | 0.2512 | 0.3114 | 0.3067 | 0.2751 | ESTs, Highly similar to |
| | | | | | | | | tropomyosin 4 [Rattus |
| | | | | | | | | norvegicus] |
| AI836045 | Mm.29976 | 0.2461 | 0.3202 | 0.2812 | 0.3016 | 0.3236 | 0.2702 | septin 5 |
| AI843823 | Mm.7414 | 0.1481 | 0.1690 | 0.1445 | 0.2971 | 0.3129 | 0.2507 | neuron specific gene family |
| | | | | | | | | member 1 |
| AI844342 | Mm.182255 | 0.1773 | 0.2039 | 0.2446 | 0.2833 | 0.3164 | 0.3083 | CD97 antigen |
| AI835331 | Mm.544 | 0.2802 | 0.3336 | 0.3057 | 0.2829 | 0.1995 | 0.2646 | phosphoprotein enriched in |
| | | | | | | | | astrocytes 15 |
| AI845602 | Mm.4146 | 0.2438 | 0.2668 | 0.3188 | 0.2727 | 0.2349 | 0.2469 | platelet derived growth |
| | | | | | | | | factor receptor, beta |
| | | | | | | | | polypeptide |
| AI838302 | Mm.4426 | 0.2816 | 0.2966 | 0.3223 | 0.2702 | 0.2466 | 0.2872 | Cd63 antigen |
| AI835546 | Mm.3117 | 0.2023 | 0.2238 | 0.2903 | 0.2696 | 0.3022 | 0.3240 | T-cell death associated |
| | | | | | | | | gene |
| AI853531 | Mm.21679 | 0.2340 | 0.3006 | 0.3272 | 0.2691 | 0.2573 | 0.2708 | RIKEN cDNA |
| | | | | | | | | 1300002F13 gene |
| AI842302 | Mm.4139 | 0.3176 | 0.3029 | 0.3261 | 0.2652 | 0.2259 | 0.2783 | rhotekin |
| AI835620 | No Data | 0.2793 | 0.3169 | 0.3180 | 0.2637 | 0.2298 | 0.2679 | No Data |
| AI845774 | Mm.856 | 0.2799 | 0.2757 | 0.3172 | 0.2630 | 0.2362 | 0.2575 | transmembrane 4 |
| | | | | | | | | superfamily member 1 |
| AI838659 | Mm.262 | 0.2496 | 0.2866 | 0.3001 | 0.2484 | 0.2192 | 0.2592 | ras homolog gene family, |
| | | | | | | | | member C |
| AI848618 | Mm.29010 | 0.1939 | 0.2150 | 0.2075 | 0.2473 | 0.2205 | 0.2216 | membrane bound C2 |
| | | | | | | | | domain containing protein |
| AI851997 | Mm.29010 | 0.2759 | 0.2851 | 0.3298 | 0.2462 | 0.2379 | 0.2648 | membrane bound C2 |
| | | | | | | | | domain containing protein |
| AI852812 | Mm.2308 | 0.2209 | 0.2669 | 0.3063 | 0.2409 | 0.2236 | 0.2485 | hemoglobin Z, beta-like |
| | | | | | | | | embryonic chain |
| AI844356 | Mm.1017 | 0.2547 | 0.2658 | 0.2582 | 0.2261 | 0.2191 | 0.2255 | esterase 10 |
| AI851647 | Mm.22240 | 0.2365 | 0.2571 | 0.2440 | 0.2219 | 0.2185 | 0.2236 | ESTs, Weakly similar to |
| | | | | | | | | SH3BGR protein |
| AI838551 | Mm.2792 | 0.1605 | 0.1832 | 0.1807 | 0.2191 | 0.1398 | 0.2238 | prostaglandin- |
| | | | | | | | | endoperoxide synthase 1 |
| AI842654 | Mm.8180 | 0.2336 | 0.2595 | 0.2941 | 0.2182 | 0.2249 | 0.2627 | lymphocyte antigen 6 |
| | | | | | | | | complex |
| AI841122 | Mm.39804 | 0.2427 | 0.2581 | 0.3048 | 0.2139 | 0.2408 | 0.2015 | EST |
| AI838653 | Mm.181074 | 0.2615 | 0.2885 | 0.3198 | 0.2073 | 0.2179 | 0.2407 | RIKEN cDNA |
| | | | | | | | | 2610001E17 gene |
| AI838959 | Mm.16537 | 0.1483 | 0.1504 | 0.2370 | 0.2014 | 0.2943 | 0.2463 | actin, alpha 2, smooth |
| | | | | | | | | muscle, aorta |
| AI842847 | Mm.8245 | 0.2013 | 0.2803 | 0.2512 | 0.1975 | 0.1770 | 0.1926 | tissue inhibitor of |
| | | | | | | | | metalloproteinase |
| AI838351 | No Data | 0.1422 | 0.1998 | 0.0999 | 0.1913 | 0.3317 | 0.2076 | No Data |
| AI837390 | Mm.43278 | 0.1418 | 0.1444 | 0.1499 | 0.1882 | 0.2873 | 0.2535 | olfactomedin related ER |
| | | | | | | | | localized protein |
| AI844326 | Mm.194675 | 0.2317 | 0.2675 | 0.2290 | 0.1847 | 0.0958 | 0.1462 | EST |
| AI839057 | No Data | 0.2107 | 0.2988 | 0.2685 | 0.1806 | 0.2179 | 0.2184 | No Data |
| AI838085 | Mm.687 | 0.1668 | 0.1773 | 0.2450 | 0.1781 | 0.2298 | 0.2301 | aplysia ras-related homolog |
| | | | | | | | | B (RhoB) |
| AI837494 | Mm.39836 | 0.1604 | 0.1709 | 0.2824 | 0.1768 | 0.1658 | 0.1247 | ESTs, Weakly similar to |
| | | | | | | | | T14318 ubiquitin-protein |
| | | | | | | | | ligase E3-alpha |
| AI836532 | Mm.196484 | 0.1481 | 0.1464 | 0.1405 | 0.1645 | 0.1642 | 0.1756 | EST AA408841 |
| AI835609 | Mm.1956 | 0.0364 | 0.0776 | 0.0791 | 0.1608 | 0.2416 | 0.1599 | neurofilament, light |
| | | | | | | | | polypeptide |
| AI842984 | Mm.980 | 0.1258 | 0.1350 | 0.1376 | 0.1602 | 0.2456 | 0.1732 | tenascin C |
| AI849378 | Mm.2769 | 0.1639 | 0.1670 | 0.1944 | 0.1545 | 0.1712 | 0.2004 | MARCKS-like protein |
| AI839275 | Mm.738 | 0.1356 | 0.1868 | 0.2704 | 0.1503 | 0.2651 | 0.1883 | procollagen, type IV, alpha 1 |
| AI844626 | Mm.29975 | 0.0684 | 0.1024 | 0.1284 | 0.1489 | 0.1956 | 0.1716 | RIKEN cDNA |
| | | | | | | | | 1810003P21 gene |
| AI835201 | Mm.8739 | 0.1115 | 0.1536 | 0.1402 | 0.1454 | 0.1709 | 0.1867 | sarcoglycan, epsilon |
| AI844312 | Mm.3091 | 0.1443 | 0.2400 | 0.2183 | 0.1432 | 0.2094 | 0.1778 | epsin 1 |
| AI841755 | Mm.687 | 0.1340 | 0.1510 | 0.1345 | 0.1427 | 0.1610 | 0.1485 | aplysia ras-related homolog |
| | | | | | | | | B (RhoB) |
| AI838813 | Mm.192516 | 0.1338 | 0.1664 | 0.1652 | 0.1416 | 0.1249 | 0.1655 | EST |
| AI839735 | Mm.37751 | 0.1409 | 0.1558 | 0.1463 | 0.1403 | 0.1138 | 0.1486 | ESTs |
| AI837031 | Mm.157662 | 0.0520 | 0.0994 | 0.1407 | 0.1260 | 0.0776 | 0.0931 | synaptotagmin 13 |
| AI840673 | Mm.29924 | 0.0846 | 0.0945 | 0.1128 | 0.1237 | 0.1111 | 0.1437 | ADP-ribosylation-like |
| | | | | | | | | factor 6 interacting protein |
| AI841538 | Mm.41009 | 0.1166 | 0.1329 | 0.2839 | 0.1210 | 0.1168 | 0.1004 | Nedd4 WW-binding |
| | | | | | | | | protein 4 |
| AI847958 | Mm.20246 | 0.1447 | 0.1526 | 0.2049 | 0.1173 | 0.0934 | 0.1017 | RIKEN cDNA |
| | | | | | | | | 2410004D18 gene |
| AI840633 | Mm.38021 | 0.0477 | 0.1194 | 0.1215 | 0.1122 | 0.0823 | 0.0391 | carbohydrate (keratan |
| | | | | | | | | sulfate Gal-6) |
| | | | | | | | | sulfotransferase 1 |
| AI843323 | Mm.3900 | 0.1334 | 0.1957 | 0.2642 | 0.1120 | 0.0902 | 0.1358 | latent transforming growth |
| | | | | | | | | factor beta binding protein 2 |
| AI849869 | Mm.34113 | 0.1241 | 0.1336 | 0.1955 | 0.1120 | 0.1015 | 0.1198 | VPS10 domain receptor |
| | | | | | | | | protein SORCS 2 |
| AI840335 | Mm.39154 | 0.0928 | 0.1347 | 0.1007 | 0.1104 | 0.1833 | 0.1133 | EST |
| AI840972 | Mm.29580 | 0.2618 | 0.3083 | 0.3024 | 0.1059 | 0.1794 | 0.1681 | superiorcervical ganglia, |
| | | | | | | | | neural specific 10 |
| AI847162 | Mm.29357 | 0.0973 | 0.0696 | 0.2018 | 0.1050 | 0.1264 | 0.1312 | RIKEN cDNA |
| | | | | | | | | 1300017C10 gene |
| AI843174 | Mm.29924 | 0.1284 | 0.1426 | 0.1479 | 0.1044 | 0.1134 | 0.1473 | ADP-ribosylation-like |
| | | | | | | | | factor 6 interacting protein |
| AI839366 | Mm.28947 | 0.0651 | 0.1159 | 0.1742 | 0.1021 | 0.1278 | 0.1395 | ESTs |
| AI840692 | No Data | 0.1394 | 0.1457 | 0.1741 | 0.0917 | 0.1456 | 0.1644 | No Data |
| AI835703 | Mm.29975 | 0.0961 | 0.0827 | 0.0714 | 0.0868 | 0.1302 | 0.1381 | RIKEN cDNA |
| | | | | | | | | 1810003P21 gene |
| AI836865 | Mm.44102 | 0.0503 | 0.0643 | 0.1129 | 0.0842 | 0.1727 | 0.1572 | ESTs |
| AI842983 | Mm.192586 | 0.0702 | 0.1325 | 0.1346 | 0.0785 | 0.1555 | 0.1091 | EST |
| AI839950 | Mm.3126 | 0.0492 | 0.0610 | 0.0989 | 0.0781 | 0.2076 | 0.1304 | four and a half LIM |
| | | | | | | | | domains 1 |
| AI844604 | Mm.3126 | 0.1263 | 0.1328 | 0.1465 | 0.0750 | 0.0188 | 0.0613 | four and a half LIM |
| | | | | | | | | domains 1 |
| AI836826 | Mm.2976 | 0.0747 | 0.0764 | 0.0755 | 0.0747 | 0.0918 | 0.0759 | glycoprotein 38 |
| AI850497 | Mm.41072 | 0.1133 | 0.1862 | 0.2509 | 0.0743 | 0.1009 | 0.0891 | ESTs, Highly similar to |
| | | | | | | | | LOX5 mouse arachidonate |
| | | | | | | | | 5-lipoxygenase |
| AI835403 | Mm.142729 | 0.0965 | 0.1012 | 0.1014 | 0.0620 | 0.0778 | 0.0579 | thymosin, beta 4, X |
| | | | | | | | | chromosome |
| AI848096 | Mm.17951 | 0.1483 | 0.1711 | 0.1888 | 0.0580 | 0.1324 | 0.1233 | erythrocyte protein band |
| | | | | | | | | 4.1-like 3 |
| AI843282 | Mm.181021 | 0.0955 | 0.1120 | 0.1453 | 0.0529 | 0.0995 | 0.1095 | procollagen, type IV, alpha 2 |
| AI842554 | Mm.192583 | 0.0577 | 0.0889 | 0.0962 | 0.0428 | 0.1088 | 0.0815 | ESTs |
| AI842681 | Mm.20904 | 0.0702 | 0.0488 | 0.1056 | 0.0405 | 0.0375 | 0.0487 | cartilage associated protein |
| AI835976 | Mm.17951 | 0.0491 | 0.0372 | 0.0362 | 0.0392 | 0.0591 | 0.0431 | erythrocyte protein band |
| | | | | | | | | 4.1-like 3 |
| AI836468 | Mm.30059 | 0.0495 | 0.0491 | 0.1289 | 0.0345 | 0.0690 | 0.0530 | myristoylated alanine rich |
| | | | | | | | | protein kinase C substrate |
| AI844038 | Mm.7919 | 0.0322 | 0.0323 | 0.0399 | 0.0339 | 0.0511 | 0.0232 | HGF-regulated tyrosine |
| | | | | | | | | kinase substrate |
| AI838614 | Mm.14802 | 0.0412 | 0.0399 | 0.0281 | 0.0331 | 0.0407 | 0.0464 | H19 fetal liver mRNA |
| AI849859 | Mm.3126 | 0.0204 | 0.0173 | 0.0375 | 0.0323 | 0.0641 | 0.0296 | four and a half LIM |
| | | | | | | | | domains 1 |
| AI837752 | Mm.43278 | 0.0346 | 0.0221 | 0.0848 | 0.0314 | 0.0460 | 0.0454 | olfactomedin related ER |
| | | | | | | | | localized protein |
| AI841798 | Mm.4871 | 0.0533 | 0.0983 | 0.1831 | 0.0273 | 0.0217 | 0.0219 | tissue inhibitor of |
| | | | | | | | | metalloproteinase 3 |
| AI838607 | Mm.4159 | 0.0277 | 0.0301 | 0.0276 | 0.0268 | 0.0559 | 0.0602 | thrombospondin 1 |
| AI842703 | Mm.147387 | 0.0284 | 0.0297 | 0.0391 | 0.0200 | 0.0205 | 0.0253 | procollagen, type III, alpha 1 |

Differentially Expressed Genes (Using Progressively Less Amine-Labeled RNA)

Since 95 genes (Table 3) were 3-fold over- or under-expressed when C2 and 3T3 cell profiles are compared using an optimal amount of total RNA (see above), it was of interest to determine how many of these genes remained 3-fold changed when progressively smaller amounts of RNA were labeled with the amine-modified primer method. C2 and NIH 3T3 RNA samples were diluted in parallel, labeled with Cy3 and Cy5, respectively, the products mixed, and one 9568-element array probed per dilution. Most of the original 95 differentially expressed genes (Table 3) were identified (i.e., ratios ≧3 or ≦1/3 between signals from the two cell lines) when 5 μg (95 genes), 2.5 μg (90 genes), and 1 μg (87 genes) of total RNA was labeled. The number of other genes not among the original 95 genes identified, but which were 3-fold changed, was fairly small (an average of 12).

With 0.5 μg of total RNA, only 72 of the differentially expressed genes were found, but the number, 11, of extraneous genes remained low. When probe was made from 0.25 μg or 0.1 μg of total RNA, there was a further decrease in differentially expressed genes detected (53 and 58, respectively), and a marked increase in false positives (71 and 97, respectively).

Analysis of Consistency of Over- or Under-Expressed Genes.

To determine how many genes will survive the above comparison when a fourth microarray is examined using the same experimental conditions, a model was studied. In the model, a log-transformed gene expression ratio, w=logt, was assumed to be normally distributed with a standard deviation of 6. For this model, the probability of observing a ratio measurement greater than 3.0 in one experiments is,

$$p = {P}_{μ = w} ( x > ln 3 ) = {\int }_{l n 3}^{∞} \frac{1}{\sqrt{2 π} σ} {ⅇ}^{- \frac{{( x - w )}^{2}}{2 {σ}^{2}}} ⅆ x \qquad ( 1 )$$

where ln(*) denotes the natural logarithm. For a ratio measurement to be greater than 3 in all of two, three or four experiments, the probabilities are simply p², p³, and p⁴, respectively. It is further assumed that within a confined ratio region [l₁, l₂], where l₁≦3≦l₂, there is equal probability for all ratio values, or pᵣ. Thus, the probability that any gene ratio within the region l₁ to l₂ is greater than 3 is given by,

$$p = {\int }_{{l}_{1}}^{{l}_{2}} {p}_{r} {P}_{μ = w} ( x > ln 3 ) ⅆ w = {p}_{r} {\int }_{w = {l}_{1}}^{{l}_{2}} {\int }_{x = l n 3}^{∞} \frac{1}{\sqrt{2 π} σ} {ⅇ}^{- \frac{{( x - w )}^{2}}{2 {σ}^{2}}} ⅆ x ⅆ w \qquad ( 2 )$$

The difference in the expected number of genes in 3 consistent experiments and 4 consistent experiments is,

$$n = N [ {\int }_{{l}_{1}}^{{l}_{2}} {{p}_{r} [ {P}_{μ = w} ( x > ln 3 ) ]}^{3} ⅆ w - {\int }_{{l}_{1}}^{{l}_{2}} {{p}_{r} [ {p}_{μ = w} ( x > ln 3 ) ]}^{4} ⅆ w ] \qquad ( 3 )$$

where N is the total number of genes within the region of [l₁, l₂]. The result for expression ratio less than 1/3 can be similarly derived. Given that the number of consistent genes were known (m=95 in this study),

$$n = m [ 1 - \frac{{\int }_{{l}_{1}}^{{l}_{2}} {[ {P}_{μ = w} ( x > ln 3 ) ]}^{4} ⅆ w}{{\int }_{{l}_{1}}^{{l}_{2}} {[ {p}_{μ = w} ( x > ln 3 ) ]}^{3} ⅆ w} ] \qquad ( 4 )$$

To numerically evaluate the above equation, a typical σ=0.07 was chosen, which can be estimated from the duplicated elements printed on the array. A typical region [l₁, l₂] was also selected, for the threshold under consideration, to be [ln(2.0), ln(4.5)]. For m=95 (3 fold changes were lumped together since Eq. 4 for over-expression and under-expression are identical). On this basis n=3.6. If σ=0.14, which is the typical variation derived from the self-on-self experiment, n=8.6. Therefore, when a 4^{th} microarray in the same experiment condition is introduced, among 95 consistently 3-fold differentially expressed genes, 4 to 9 genes are expected to be dropped due to random variation of the microarray assay. In other words, 90 and 87 genes obtained from 2.5 μg and 1 μg were within the expectation, thus their experiment conditions should be comparable, although the amount of RNA used to make probe was different. For less input RNA (from which 72 or less genes in the differentially expressed class were detected), the number is far below that expected, and it is concluded that insufficient RNA was employed.
Modified Random Primer Labeling Shows No Cyanine Label Bias

In all of the reported studies with the new labeling techniques, only 1-5 μg or less total RNA was used as template. In spite of the low amount of total RNA used, this system produces highly reliable and consistent data.

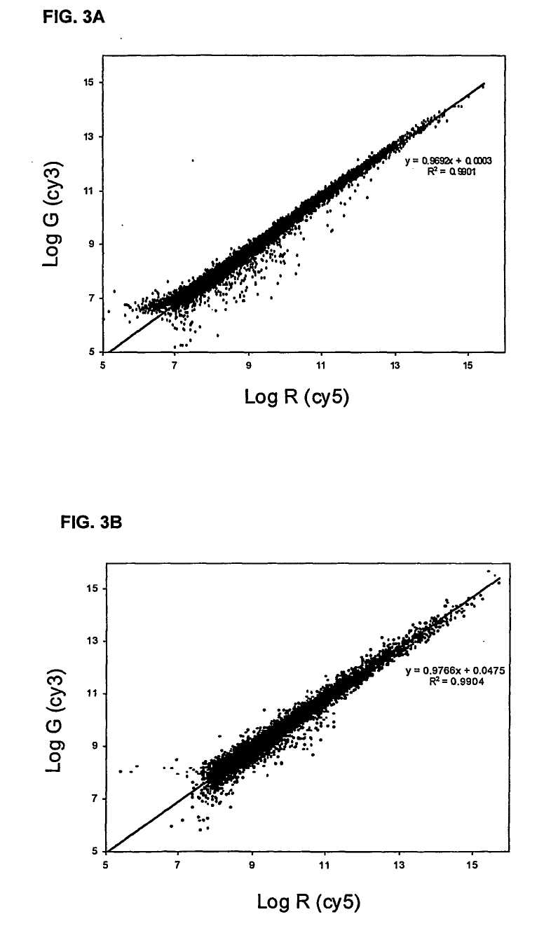

To test the labeling and hybridization signal reliability of the new labeling method, the same amount RNA was labeled (5 μg mouse C2 cell line total RNA) with two different dyes (Cy3 and Cy5) to generate Cy5 and Cy3-labeled probes. The two probes were hybridized to the arrays and scanned (photomultiplier tube (PMT) voltages of 600 and 550 for Cy5 and Cy3, respectively. Scatter plots of log intensity Cy5 signal versus log intensity Cy3 signal and log (Cy5/Cy3) versus Average log intensity are shown. Data shows (FIG. 3A) that the two probes generated similar signal intensities though they were labeled by two different dyes.

Cy5 and Cy3-labelled probes were also prepared from 5 μg and 1 μg of total C2 RNA, respectively. PMT voltages of 600 and 580 were used to scan the Cy5 and Cy3 channels. These signals were strongly correlated (FIG. 3B).

A recent study using traditional labeling techniques (Taniguchi et al., Genomics 71, 34-39, 2001) clearly showed the inconsistency of labeling and hybridization results from the reverse combination of dyes, due to the bias of dye labeling. In that study, a notably larger quantity of template RNA was required for Cy5 labeling when the traditional direct labeling method was used. The modified random primer labeling system reported herein overcomes this labeling bias.

P1 Versus P2, Same Amount of Template RNA

Another experiment was carried out to compare hybridization signals from probe labeled with amine modified random primer P2 and regular random primer P1, using 5 μg total RNA from mouse C2 cell line for both labeling methods and both Cy3 and Cy5 labeling.

The two probes labeled using two different primers were hybridized to each of two identical arrays on the same slide, as described above. The slide was then scanned at same laser power and PMT level (620 volts and 600 volts for the Cy5 and Cy3 channels, respectively). The images were processed and analyzed with IPLab/ArraySuite. The hybridization intensity from the array hybridized with probe labeled with primer P2 were substantially stronger than the intensity achieved from probe labeled with primer P1.

These comparison data were quantified and indicate that hybridization intensities using P2 labeling were at least 2.5 fold higher than P1. Amine modified random primer P4 showed similar results. Thus, with more amine groups being incorporated into the probes using the modified random primers, the fluorescent signals are demonstrated to be much higher when using an equivalent amount of starting template.

This reveals that, when comparing the traditional random primer (P1) with an amine modified random primer (P2), the signal from incorporating a primer with a single amine (—NH₂) group into each cDNA (using P2) is roughly equivalent to that achieved when amine modified base is included only in the RT reaction (using P1). Thus, roughly only 1-2 amine labeled nucleotides are incorporated by RT per strand synthesized; this suggests that synthesis may cease once a single modified nucleotide is incorporated. Therefore, one strategy for increasing incorporation is not by adding amine- or dye-modified nucleotides in the RT reaction, but by adding additional modifications to the primer. For this reason, also provided are additional modified primers (SEQ ID NOs: 4-9, for instance) comprising two (or more) modified bases (e.g. amine-modified bases), which optionally may be separated by 0-5 inosine residues. Signal intensity from the label molecule may vary depending on the spacing between multiple modified bases within a single primer.

Sensitivity and Clone Detection

The modified primer labeling method increased hybridization sensitivity as well. Starting with the same amount of template RNA, probes labeled using amine modified random primer P2 could detect about 60 genes that were not detectable with probes labeled using regular random primer P1.

A recent study (Taniguchi et al., Genomics 71, 34-39, 2001) demonstrated that some genes were not detectable using standard DNA microarrays when compared with conventional Northern blot analysis. This defect in the traditional labeling method may be overcome using the modified random primer labeling methods disclosed herein.

In distinct contrast to prior labeling methods, the modified primer labeling methods, as demonstrated here with amine modified random primer, produce significant signals and increases sensitivities, whether the probe is labeled with Cy3 or Cy5. Much less RNA is required for making high quality probes and there is no bias of dye incorporation using same amount of RNAs for both Cy3 and Cy5 labeling.

### Example 4

### Amplification Coupled with Amine modified random primer Labeling (Method 1)

The disclosed amine modified random primers can also be used with T7-mediated amplification of transcript, to further reduce the amount of starting material necessary to produce a hybridization probe. This can be carried out using the following protocol:

I. cDNA Synthesis

First strand synthesis is carried out using the following Primer-RNA mixture:

Primer-RNA Mix

| | |
|---|---|
| Total RNA (less than 1 μg) | 1-5 μl |
| DEPC water | variable |
| T7 - Oligo dT primer (100 pm/μl) | 1 μl |
| Total | 10 μl |

This mixture is incubated at 70° C. for 10 minutes, then chilled on ice 10 minutes to facilitate annealing of the primer to the template.

To each reaction is then added 10 μl of the following reverse transcription mixture:

RT Mix

| Component | μl | For 10 reactions (10.2 fold) |
|---|---|---|
| 5X first strand buffer | 4 | 40.8 |
| 10 mM dNTPs | 1 | 10.2 |
| DTT (0.1 M) | 2 | 20.4 |
| DEPC water | 1 | 10.2 |
| SSII RT | 2 | 30.6 |
| Total | 10 | |

The first strand of cDNA is synthesized by incubating the tubes at 42° C. for 2 hours.

To initiate second strand synthesis, the following reagents are mixed with a first strand synthesis reaction:

| | |
|---|---|
| RNase-free water | 91 μl |
| 5X second strand buffer | 30 μl |
| 10 mM dNTPs | 3 μl |
| E. coli DNA ligase | 1 μl |
| E. coli DNA polymerase I | 4 μl |
| RNase H | 1 μl |
| Total (including first strand reaction) | 150 μl |

The reaction mixture is then incubated at 16° C. for 2 hours. A 2 μl aliquot of T4 DNA polymerase is added, and the mixture incubated at 16° C. for 5 minutes. The reaction is stopped by adding 10 μl of 0.5 M EDTA (pH 8.0)

The double stranded cDNA (ds cDNA) is then extracted, for instance using Phase Lock Gel (PLG) extraction, using the manufacturer's instructions. To prime it, the PLG tube is pelleted by centrifuging for 30 seconds at maximum speed in a microfuge. The ds cDNA (approximately 162 μl) is mixed with an equal volume of Phenol-Chloroform-IAA (162 μl) and vortexed. All of this mixture is added to the PLG tube, and the tube centrifuged for two minutes at maximum speed.

The resulting ds cDNA preparation can be further cleaned up using for instance, a Microcon 100 concentrator from Amicon. The Microcon 100 is filled with 400 μl dd-H₂O, and the top aqueous layer from above PLG extraction transferred into it. The column is then spun at maximum speed for about 2 minutes (or until about 20 μl left), and the flow-through discarded. This washing process is repeated twice more with 500 μl dd-H₂O. The concentrated and cleaned ds DNA sample is collected by inverting the tube and spinning at 5000 rpm for 5 minutes. The resultant sample is dried in a speed vacuum, and re-suspended in 4.5 μl of RNase-free water.

II. In Vitro Transcription

Double-stranded cDNA produced as above is then used in an in vitro transcription reaction, using for instance an Ambion in vitro transcription (IVT) kit, as follows:

The IVT reaction comprises the following:

| | |
|---|---|
| Ambion T7 10X ATP | 2 μl |
| Ambion T7 10X GTP | 2 μl |
| Ambion T7 10X UTP | 2 μl |
| Ambion T7 10X CTP | 2 μl |
| RNase-free water | 3.5 μl |
| ds DNA synthesized above | 4.5 μl |
| 10X T7 transcription buffer | 2 μl |
| 10X T7 enzyme mix | 2 μl |
| Total | 20 μl |

The transcription reaction is incubated at 37° C. for six hours

In vitro transcribed RNA made in this manner can be cleaned up, for instance, using Qiagen RNeasy mini columns and protocols as supplied by the manufacturer, essentially as follows:

In 1.5 ml tube, the following reagents are mixed:

| | |
|---|---|
| RNase free water | 80 μl |
| IVT reaction | 20 μl |
| Buffer RLT (supplied with kit) | 350 μl |
| 100% EtOH | 250 μl |

The mixture (700μ) is vortexed gently to mix, then placed in the RNeasy column, where it is incubated for two minutes to provide time for the RNA to bind to column. The column is then centrifuge at 2000 rpm for 5 minutes, and the flow-through reserved. The column is washed (twice) with 500 μl of RPE (with EtOH added), and centrifuge at 10K for 1 minute. The column is then centrifuged at maximum speed for 1 minute to remove any remaining fluid, and RNA packed column into a new 1.5 ml collection tube. RNase-free water (30 μl) is added, and the tube incubated for 1-2 minutes. The column is centrifuged at 5000 rpm for 5 minutes, then 10K for 30 seconds, and the eluate collected. The elution process repeated with an additional 30 μl of RNase-free water, for a final eluate volume of approximately 60 μl. The copy RNA (cRNA) yield can be quantitated by measuring its optical density (OD) using standard techniques.
III. Labeling with Modified Random Primer Using cRNA as Template

cRNA produced as above can be used as the template for production of labeled probe molecules using the modified (e.g., amine modified) random primers provided herein.

A 17 μl aliquot of the RT mix (6 μl 5× first strand buffer (provided with SSII RT), 6 μl 3× aa-dUTP/dNTPs, 3 μl 0.1M DTT, and 2 μl SuperScript II Reverse transcriptase (SSII RT; GIBCO BRL Life Technologies, Rockville, Md.)) is added to each primer-RNA mix, for a total volume of 30 μl, and the sample incubated at 42° C. for two hours to permit reverse transcription of the cDNA.

The reverse transcription reaction was stopped by the addition of 10 μl of 0.5M EDTA. RNA was degraded from the reaction mixture by adding 10 μl of 1N NaOH, and incubating the sample at 65° C. for 30 minutes. The reaction was neutralized by adding 10 μl of 1M HCl.

The neutralized cDNA sample was cleaned up using a MicroCon 100 concentrator cartridge (Millipore, Bedford, Mass.). The cartridge was primed with 450 μl of ddH₂O, then the neutralized reaction added and the cartridge spun at 13,000 rpm for about 3 minutes. The flow-through was discarded, and the cDNA on the cartridge washed twice with 500 μl of ddH₂O. The cDNA sample was eluted from the cartridge, and dried in a speed vacuum.

IV. Coupling, Quenching and Cleanup

The cDNA sample is resuspended in 4.5 μl water. Pre-dried aliquots of monofunctional NHS-ester Cy3 or Cy5 dye (prepared as in Example 2) resuspended in 4.5 μl of 100 mM NaBicarbonate Buffer (pH 9.0). One aliquot of cDNA is mixed with one aliquot of Cy3 or Cy5, and the samples incubated at RT for 1 hour in dark to couple the fluorescent dye to the modified cDNA.

The fluorescence is quenched by adding 4.5 μl of 4M hydroxylamine to the dye-coupled cDNA, and incubating the mixture at RT for 30 minutes in dark. The labeled sample is cleaned up using a Qiagen Qia-quick PCR purification kit (Qiagen, Valencia, Calif.) as described in Example 2.

Hybridization to microarrays and analysis of the resultant data are carried essentially as described above in Example 3.

### Example 5

### Amplification Coupled with Amine Modified Random Primer Labeling Method 2)

In another embodiment, asRNA is amplified using the following protocol. Total RNA is isolated from a biological sample, such as a fresh or preserved cell or tissue sample or an aliquot of cells grown in culture. By way of example, total RNA is isolated using a Qiagen midi kit (Cat. #75142) following the instructions provided by the manufacturer. Alternatively, Trizol extraction (Gibco BRL Cat. # 15596-026) could also be used (following the procedures provided by the manufacturer). The total RNA is then resuspended or eluted in DEPC water.

First strand cDNA synthesis is carried out as follows: In a PCR reaction tube, 0.001-3 μg total RNA is mixed in 9 μl DEPC H₂O with 1 μl (0.01-0.5 μg/μl) oligo dT₍₁₅₎-T7 primer (SEQ ID NO: 11) and heated to 70° C. for three minutes, then cooled to room temperature. To this is then added the following reagents (which can be made into a “mastermix” for multiple samples):
4 μl 5× First strand buffer (provided with Superscript II kit)
1 μl (0.1-0.5 μg/μl) TS (template switch) oligo primer (SEQ ID NO: 3)
2 μl 0.1M DTT
1 μl RNasin (Promega Cat. # N2111)
2 μl 10 mM dNTP (Pharmacia Cat. # 27-2035-O₂)
2 μl Superscript II polymerase (Gibco BRL Cat # 18064-071)
   The reaction is then incubated 42° C. for 90 minutes, for instance in a thermal cycler.

Second strand synthesis is carried out by adding the following reagents to each cDNA reaction tube:
106 μl of DEPC H₂O
15 μl Advantage PCR buffer
3 μl 10 mM dNTP
1 μl of RNase H (2 U/μl, Gibco BRL Cat# 18021-071)
3 μl Advantage Polymerase (CLONTECH Cat# 8417-1)
   The samples are then incubated at 37° C. for five minutes to digest mRNA, 94° C. for two minutes to denature, 65° C. for one minute for specific priming, and 75° C. for 30 minutes for extension of the second strand. The reaction is stopped by adding 7.5 μl 1M NaOH solution containing 2 mM EDTA and incubating at 65° C. for 10 minutes to inactivate enzyme.

The double stranded (ds) cDNA can be cleaned up as follows: A 1 μl aliquot of Linear Acrylamide (0.1 μg/μl, Ambion Cat. # 9520) is added to each sample. The sample is then extracted by adding 150 μl Phenol: Chloroform: Isoamyl alcohol (25:24:1) (Boehringer Mannheim Cat. #101001) to each ds cDNA tube and mixing well by pipetting. It is important to be careful not to spill or contaminate the sample. The slurry solution is then transferred to Phase lock gel tube (5′-3′ Inc. Cat. # p1-257178) and spun at 14,000 rpm for five minutes at room temperature. The aqueous phase is transferred to RNase/Dnase-free tube and 70 μl of 7.5M ammonium acetate (Sigma Cat# A2706) added, followed by 1 ml 100% ethanol. This tube is centrifuged at 14,000 rpm for 20 minutes at room temperature to pellet the nucleic acid. The resultant pellet is washed twice with 500 μl 100% ethanol and spun down at maximum speed for eight minutes. Finally, the ds cDNA pellet is air dried and resuspended in 70 μl DEPC H₂O.

Bio-6 Chromatograph columns (Bio-Rad Cat. # 732-6222) are prepared by washing the columns with 700 μl DEPC H₂O three times and spinning at 700×g for two minutes at room temperature. (It may be important to shake the washed column well before draining to get rid of air bubbles—otherwise it drains very slowly.) When opening the column, any gel in the underside of the cap is aspirated off to prevent contamination. Also, the collection tubes provided with Bio-6 columns are not RNase-free; the samples should be collected in RNase-free tubes.

For each sample, 70 μl is loaded onto the center of the column and the column spun at 700×g for four minutes. The sample is then dried by Speedvac and resuspended in 8 μl DEPC water.

Using this double-stranded cDNA, in vitro transcription (IVT) is performed using an Ambion T7 Megascript Kit (Cat. #1334). For each sample, the following reaction mixture is made:
2 μl of each 75 mM NTP (A, G, C and UTP)
2 μl reaction buffer
2 μl enzyme mix (RNase inhibitor and T7 phage polymerase)
8 μl ds cDNA (produced as described herein)
   The reactions are then incubated at 37° C. for six hours to permit transcription.

The asRNA produced is then purified using TRIzol reagent (GibcoBRL, Cat. #15596). To each IVT reaction is added 1 ml of TRIzol solution, and the tubes are mixed well. 200 μl of chloroform is then added per 1 ml TRIzol solution, and the samples mixed by inverting for 15 seconds. They are then incubated at room temperature for 2-3 minutes, and centrifuged at 12,000 g for 15 minutes at 4° C. The aqueous phase is then transferred to a new RNase free tube and 500 μl of isopropyl alcohol added per 1 ml TRIzol reagent to precipitate the nucleic acids. The samples are incubated at room temperature for 10 minutes and then centrifuged at 14,000 rpm for 15 minutes. The resultant pellet is washed two times with 1 ml 70% ethanol in DEPC-treated water, the pellet air dried and quickly resuspended in 20 μl DEPC-treated water. (Over-dried RNA is difficult to dissolve into water). RNA concentration can be checked and quality estimated by measuring OD₂₆₀ and OD₂₆₀/₂₈₀ using standard techniques.

An RNA easy mini kit also could be used to recover the asRNA (but the recovery of asRNA may be lower that that achieved with the TRIzol method.)

The asRNA may be subjected to a second round of amplification, though this is not necessary in all embodiments. By way of example, asRNA (0.5-1 μg) produced as above is mixed in 9 μl DEPC H₂O with 1 μl (2 μg/μl) random hexamer (i.e., dN₆) and heated to 70° C. for three minutes, then cooled to room temperature. The following reagents are then added:

4 μl 5× First strand buffer
1 μl (0.5 μg/μd) oligo dT-T7 primer
2 μl 0.1M DTT
1 μl RNasin (Promega Cat # N2111)
2 μl 10 mM dNTP (Pharmacia Cat. # 27-2035-O₂)
2 μl Superscript II (SS II) (Gibco BRL Cat # 18064-071)
   The samples are then incubated at 42° C. for 90 minutes. The resultant single-stranded cDNA then can be subjected to second strand synthesis and cleanup similarly to that described above. By way of example, the ds cDNA is then resuspended in 16 μl of DEPC treated water.

Second round in vitro transcription (IVT) proceeds using the following reaction mixture:
4 μl of each 75 nM NTP (A, G, C and UTP)
4 μl reaction buffer
4 μl enzyme mix (RNase inhibitor and T7 phage polymerase)
16 μl ds cDNA
   Each reaction is incubated at 37° C. for six hours, and the asRNA purified using TRIzol reagent, as described above.

asRNA amplified from the second IVT then can be converted into cDNA using modified random primers as provided herein and reverse transcription, for instance using the following reaction:
6 μg of asRNA (1 μg/μl)
2 μl of modified random primer (8 μg/μl)
14 μl of DEPC treated water
   Samples are heated to 70° C. for three minutes and then put on ice. Then, the following reagents are added:
8 μl of 5× first strand buffer
4 μl of 10 mM dNTP (with or without the addition of aa-dNTP as described herein)
4 μl of 0.1M DTT
2 μl of RNasin
3 μl of Superscript II
   The samples are then incubated at 42° C. for 90 minutes. The reactions are stopped by adding 5 μl of 0.5M EDTA with 10 μl of 1M NaOH and heating to 65° C. for 10 minutes, which hydrolyzed the asRNA and inactivated the enzymes. The pH of the samples is neutralized by adding 25 μl of 1M Tris pH 7.5.

Target nucleic acids may be purified (precipitated) as follows: To each sample is added 30 μl of ammonium acetate and 500 μl 100% ethanol, and the samples are mixed and incubated at −20° C. for 15 minutes. Samples are centrifuged at 13,000 rpm at 4° C. for 20 minutes, and the resultant pellet washed twice with 500 μl of 70% ethanol. The pellet is then completely dried using a Speedvac, and the purified cDNA resuspended in 12.5 μl of 3×SSC; in some embodiments, to get a stronger signal the cDNA is resuspended in a smaller volume. Resuspended cDNA can be stored at −20° C. prior to labeling with a detectable molecule, such as a Cy3 or Cy5.

### Example 6

### RNA Amplification with T3N9 Primers Coupled with Amine Modified Random Primer Labeling (Method 3)

The disclosed primer modification (such as amine modification) can be used with T3N9 primer-mediated amplification of transcript to produce a collection of RNA species. An advantage of using the T3N9 primer is that, unlike transcripts generated with random hexamers and T7-oligo dT primers, transcripts generated with T3N9 primers are substantially less 3′ biased. As a result, the length of T3N9 primer-mediated transcripts tends not to decrease with each round of amplification. By way of example, amplification using T3N9 primers can be carried out using the following protocols:

I. RNA Production

Amplified RNAs were prepared either from total RNA sources or directly from cells.

If starting with cells, BCBL1 and 293 cells were collected and washed in cold 1×PBS (Invitrogen, Carlsbad, Calif.). The cells were counted and diluted to a density of 5000 cells/ml. Two μl of cells (˜10 cells) were added to a 0.5 ml tube containing a mixture of 6 μl of 5× first strand buffer (Invitrogen, Carlsbad, Calif.), 31 μl of RNase-free water (Invitrogen, Carlsbad, Calif.), and 1 μl of RNase inhibitor (Promega, Madison, Wis.). The cells were broken apart by sonication. After spinning at 13,000 rpm at 4° C. for 15 minutes, the supernatant was transferred to a 0.2 ml PCR tube and concentrated to 23 μl with a SpeedVac (Thermo Savant, Holbrook, N.Y.). In order to digest the genomic DNA, 0.5 μl of Dnase I (Ambion, Austin, Tex.) was added to the sample, then incubated for 30 minutes at 37° C. The Dnase I was inactivated by incubating the sample at 75° C. for 5 minutes.

If starting with total RNA, human BCBL1 and 293 cells were collected and total RNA was extracted using TRIzol reagent from Invitrogen Life Technologies (Carlsbad, Calif.) following the manufacturer's instructions. Two μl of total RNA (0.5 μg) was added in a 0.2 ml PCR tube containing 6 μl of 5× first strand buffer, 31 μl of RNase-free water, and 1 μl of RNase inhibitor. The sample was concentrated to 23 μl before initiating the first strand cDNA synthesis.

II. cDNA Synthesis

T7-oligo dT primer (SEQ ID NO: 13) from Operon (Alameda, Calif.) (1 μl, at a concentration of 100 pmol/μl) was added to 23 μl of total RNA or the RNA derived from the 10 cells, as described above. The RNA was denatured at 70° C. for 10 minutes and chilled, on ice, for 10 minutes. 1 μl of 10 mM dNTPs (Amersham Pharmacia, Piscataway, N.J.), 3 μl of 0.1 mM DTT (Invitrogen, Carlsbad, Calif.) and 2 μl of SuperScript II reverse transcriptase (Invitrogen, Carlsbad, Calif.) were added to the tube, and the reaction mixture was incubated at 42° C. for 2 hours to carry out first strand cDNA synthesis.

For second strand cDNA synthesis, 81 μl of RNase-free water, 30 μl of 5× second strand buffer (100 mM Tris-HCl, pH 6.9; 450 mM KCl; 23 mM MgCl₂; 0.75 mM beta-NAD⁺; and 50 mM (NH₄)₂SO₄), 3 μl of 10 mM dNTPs, 1 μl of E. coli DNA ligase (Invitrogen, Carlsbad, Calif.), 4 μl of E. coli DNA polymerse I (Invitrogen, Carlsbad, Calif.), and 1 μl of RNase H (Invitrogen, Carlsbad, Calif.) were added to bring the total volume of the sample to 150 μl. The reaction was then incubated for 2 hours at 16° C. Following the incubation, 2 μl of T4 DNA polymerase (Invitrogen, Carlsbad, Calif.) was added to the sample, followed by a 5 minute incubation at 16° C.

Phase Lock Gel (Eppendorf, Westbury, N.Y.) and phenol-chloroform-IAA (Invitrogen, Carlsbad, Calif.) were used to extract the cDNA using the manufacturer's protocol. The sample was then applied to a MicroCon-30 column (Millipore, Bedford, Mass.) to further clean and concentrate the cDNA. The cDNA was dried in a SpeedVac and resuspend in 4.5 μl of RNase-free water.

III. RNA Amplification

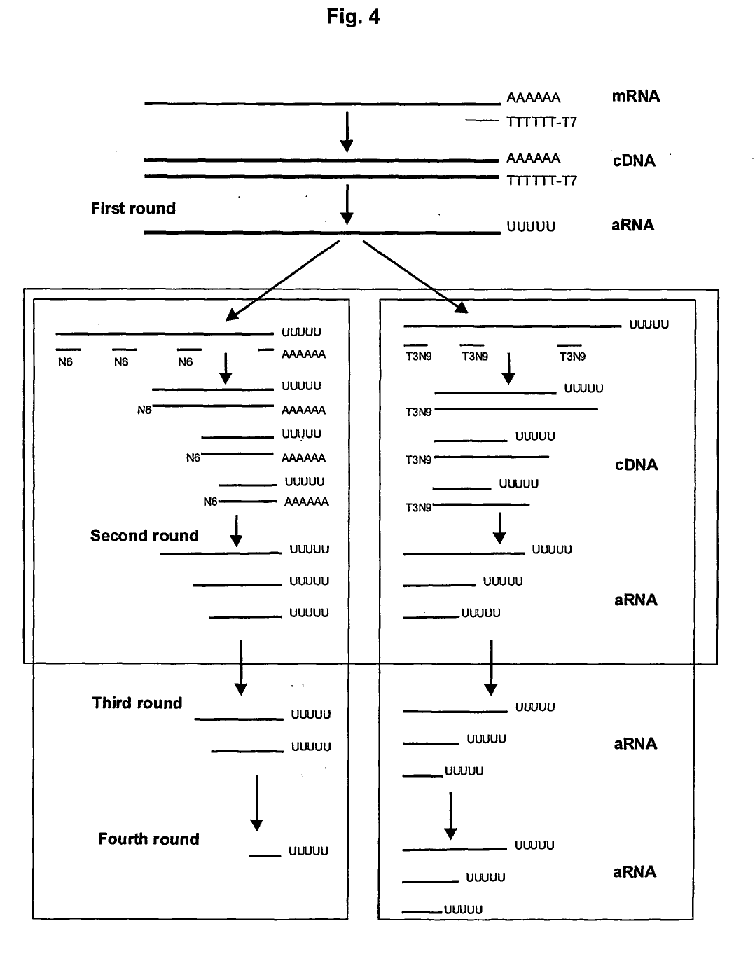

First round amplified RNA was then transcribed from the double-stranded cDNA with MEGAscript T7 kit (FIG. 4) (Ambion, Austin, Tex.), according the manufacturer's instructions, followed by clean-up with RNeasy Mini kit (Qiagen, Valencia, Calif.).

The second and subsequent rounds of amplification were carried out using two different methods (FIG. 10). One method was essentially as described by Wang et al., Nat. Biotechnol. 18, 457-459 (2000). Specifically, 0.5-1 μg first round amplified RNA was mixed with 1 μl of random hexamer (dN6) (2 μg/μl) in 9 μl DEPC water. The mixture was heated to 70° C. for 3 minutes, then cooled to room temperature. The following reagents were added to the mixture: 4 μl 5× first strand buffer (provided with Superscript II), 1 μl (0.5 μg/μ) oligo dT-T7 primer, 2 μl 0.1M DTT, 1 μd RNAsin (Promega Cat# N2111), 2 μl 10 mM dNTP (Pharmacia Cat# 27-2035-02), and 2 μl Superscript II (SS 10) (Gibco BRL Cat# 18064-071). The mixture was incubated at 42° C. for 90 minutes. Second strand cDNA synthesis and double stranded cDNA cleanup were performed as described above. In the second round of in vitro transcription, 40 μl of the in vitro transcription reaction mixture was used instead of 20 μl. RNA isolation followed, as described above.

The second method of second and subsequent rounds of amplification used a custom designed T3N9 primer (SEQ ID NO: 12) (Invitrogen, Carlsbad, Calif.) for priming both the first strand cDNA and second strand cDNA synthesis. Specifically, 17 μl of first round amplified RNA was mixed with 1 μl of T3N9 (100 pm/μl) and the mixture was incubated at 70° C. for 10 minutes then chilled, on ice, for 10 minutes. The following reagents were then added to the mixture: 6 μl 5× first strand buffer, 1 μl of 10 mM dNTPs (Amersham Pharmacia, Piscataway, N.J.), 3 μl of 0.1 mM DTT (Invitrogen, Carlsbad, Calif.) and 2 μl of SuperScript II reverse transcriptase (Invitrogen, Carlsbad, Calif.) were added to the tube, and the reaction mixture was incubated at 42° C. for 2 hours to carry out first strand cDNA synthesis. Second strand cDNA synthesis, double stranded cDNA clean-up and subsequent in vitro transcription were performed as described above.

IV. Probe Labeling Using Amine Modified Random Primers

The amplified RNA can be used as a template for production of labeled probe molecules using the modified (e.g., amine modified) primers, as described in Examples 4 and 5 above. Five μg of total RNA or 2 μg of amplified RNA (5 μg for the amplified RNA obtained directly from cells) were used for labeling the cDNA probes.

V. cDNA Microarrays

Amplified RNA generated from 1-4 rounds of amplification with the T3N9 primer, as described above, and total RNA, were obtained from human BCBL1 and 293 cell lines. Using the total RNA or the amplified RNA as templates, cDNA probes were generated with the amine modified random primers, as described in Examples 4 and 5, above. The probes were then hybridized to the microarrays as follows: the cDNA probes were partially dried in a vacuum centrifuge to a volume of 17 μl and to the DNA was added 1 μl of poly A (8 mg/ml), 1 μl of Cot-1 DNA (10 mg/ml) and 1 μl of yeast tRNA (4 mg/ml). The probe mixture was denatured at 98° C. for 2 minutes, chilled on ice and 20 μl of the probe mixture was mixed with 20 μl of 2× F-Hybridization buffer (250 μl of 100% formamide, 250 μl of 20×SSC, 10 μl of 10% sodium dodecyl sulfate). An aliquot of the mixture (35 μl) was applied to arrays. The arrays were covered with 22×60 mm coverslips and then incubated overnight, in a water bath, at 42° C. Following the incubation, the cover-slips were removed from the arrays while they were soaking in pre-wash buffer (2×SSC, 0.1% sodium dodecyl sulfate) and the arrays were washed for 5 minutes at room temperature in first wash buffer (0.5×SSC, 0.01% sodium dodecyl sulfate) followed by a wash with second wash buffer (0.06×SSC) for 5 minutes at room temperature. The arrays were dried by spinning them in a centrifuge at 800 rpm for 2 minutes.

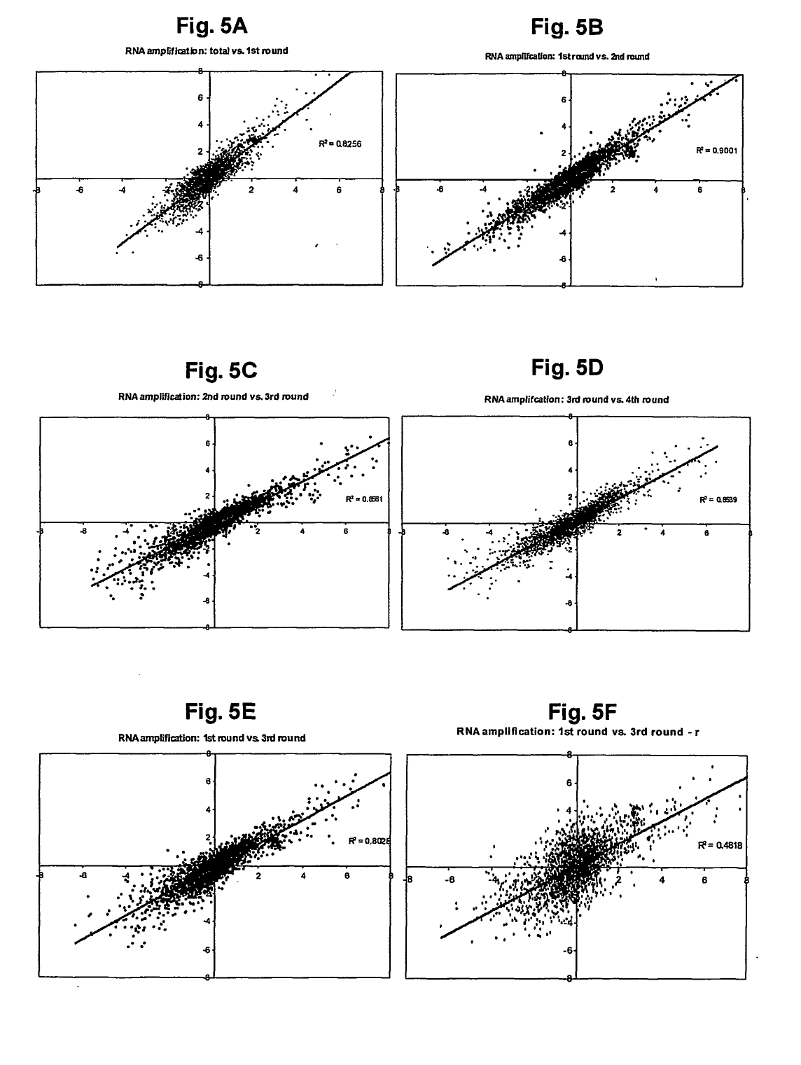

All experiments used 6500 element human cDNA arrays. The ratios were determined by comparing the intensities, captured with a laser scanner, of the BCBL1 and 293 cell lines (FIG. 5). A strong correlation was observed between (FIG. 5A) total RNA/first round amplification, (FIG. 5B) first/second round amplification, (FIG. 5C) second/third round amplification, and (FIG. 5D) third/fourth round amplification, with R²=0.8256, 0.9001, 0.8561, and 0.8539, respectively. A good correlation was also demonstrated in FIG. 5E after three rounds of amplification using the T3N9 primers (12=0.8018) compared to the standard method, as shown in FIG. 5F, that uses random hexamers and T7-oligo dT primers (R²=0.4818).

V. Differentially Expressed Genes

In one specific experiment, cultured mouse C2 and NIH 3T3 cells were diluted to a density of 10 or 100 cells per sample. First strand cDNA synthesis directly from cells, second strand cDNA synthesis and RNA amplification were performed as described above. After three rounds of amplification, approximately 20 μg of amplified RNA was obtained Half of the amplified RNA was used in the labeling reaction. The microarray expression patterns were similar between the total RNA and aRNA and the RNA amplified from 10 and 100 single cells. Genes with a 3-fold or greater difference in expression were identified (73 genes) which was comparable to the number of genes identified (90 genes) with total RNA. The most differentially expressed genes are listed in Table 4.

TABLE 4

| Total RNA | 10cellAmp3rd | Clone ID | Description |
|---|---|---|---|
| 18.3218 | 9.6548 | AI849214 | whey acidic protein |
| 6.6261 | 4.1602 | AI836864 | forkhead box G1 |
| 6.2954 | 3.375 | AI838361 | Mus musculus 10 days embryo cDNA, RIKEN full- |
| | | | length enriched library, clone: 2610305D13, full |
| | | | insert |
| 5.528 | 6.2676 | AI843085 | RIKEN cDNA 5730403B10 gene |
| 5.5015 | 4.6931 | AI842716 | cytochrome P450, 51 |
| 5.2438 | 4.4078 | AI847098 | ERO1-like (S. cerevisiae) |
| 5.225 | 7.4932 | AI846827 | Mus musculus, Similar to oxidation resistance 1, |
| | | | clone MGC: 7295, mRNA, complete cds |
| 4.0612 | 12.1508 | AI840688 | transketolase |
| 3.7839 | 6.6078 | AI852317 | N-myc downstream regulated 1 |
| 3.7376 | 3.2635 | AI843677 | Erbb2 interacting protein |
| 3.715 | 3.6818 | AI844828 | glycine transporter 1 |
| 3.2641 | 3.0637 | AI847571 | matrin 3 |
| 3.0989 | 8.5314 | AI841304 | EST |
| 3.0926 | 11.1423 | AI838612 | glutathione S-transferase, mu 2 |
| 3.0799 | 3.7138 | AI847962 | transmembrane 4 superfamily member 2 |
| 3.0628 | 3.2787 | AI839363 | mammary tumor integration site 6 |
| 0.2926 | 0.0848 | AI846190 | ATPase, H+ transporting, lysosomal (vacuolar |
| | | | proton pump), alpha 70 kDa, isoform 2 |
| 0.2816 | 0.3131 | AI838302 | Cd63 antigen |
| 0.2799 | 0.1641 | AI845774 | transmembrane 4 superfamily member 1 |
| 0.2793 | 0.2697 | AI835620 | No Data |
| 0.2668 | 0.1074 | AI851985 | RIKEN cDNA 2610024P12 gene |
| 0.264 | 0.1284 | AI840752 | cAMP responsive element binding protein 3 |
| 0.2615 | 0.2032 | AI838653 | RIKEN cDNA 2610001E17 gene |
| 0.2547 | 0.1201 | AI844356 | esterase 10 |
| 0.2365 | 0.1955 | AI851647 | ESTs, Weakly similar to SH3B_MOUSE SH3 |
| | | | DOMAIN-BINDING GLUTAMIC ACID-RICH |
| | | | PROTEIN (SH3BGR PROTEIN) |
| 0.2336 | 0.3206 | AI842654 | lymphocyte antigen 6 complex |
| 0.2151 | 0.1776 | AI836265 | ESTs |
| 0.2107 | 0.1573 | AI839057 | No Data |
| 0.2013 | 0.185 | AI842847 | tissue inhibitor of metalloproteinase |
| 0.1946 | 0.1997 | AI853210 | procollagen, type IV, alpha 1 |
| 0.1791 | 0.3161 | AI834944 | RIKEN cDNA 5530400B01 gene |
| 0.1639 | 0.1518 | AI849378 | MARCKS-like protein |
| 0.1605 | 0.1248 | AI838551 | prostaglandin-endoperoxide synthase 1 |
| 0.1604 | 0.1033 | AI837494 | ESTs, Weakly similar to T14318 ubiquitin-protein |
| | | | ligase E3-alpha - mouse [M. musculus] |
| 0.1556 | 0.1074 | AI840347 | EST |
| 0.1513 | 0.1874 | AI842286 | protein tyrosine phosphatase, receptor type, K |
| 0.1492 | 0.1851 | AI836264 | tissue inhibitor of metalloproteinase 3 |
| 0.1483 | 0.0755 | AI848096 | erythrocyte protein band 4.1-like 3 |
| 0.1447 | 0.2196 | AI847958 | RIKEN cDNA 2410004D18 gene |
| 0.1422 | 0.3007 | AI838351 | No Data |
| 0.1394 | 0.1511 | AI840692 | No Data |
| 0.1356 | 0.1468 | AI839275 | procollagen, type IV, alpha 1 |
| 0.1338 | 0.0426 | AI838813 | EST |
| 0.1284 | 0.0588 | AI843174 | ADP-ribosylation-like factor 6 interacting protein |
| 0.1263 | 0.0146 | AI844604 | four and a half LIM domains 1 |
| 0.1258 | 0.2362 | AI842984 | tenascin C |
| 0.1166 | 0.0592 | AI841538 | Nedd4 WW-binding protein 4 |
| 0.1133 | 0.2358 | AI850497 | ESTs, Highly similar to LOX5 MOUSE |
| | | | ARACHIDONATE 5-LIPOXYGENASE |
| | | | [M. musculus] |
| 0.1115 | 0.1109 | AI835201 | sarcoglycan, epsilon |
| 0.1031 | 0.1129 | AI845475 | ESTs |
| 0.0965 | 0.1415 | AI835403 | thymosin, beta 4, X chromosome |
| 0.0961 | 0.1652 | AI835703 | RIKEN cDNA 1810003P21 gene |
| 0.0955 | 0.1569 | AI843282 | procollagen, type IV, alpha 2 |
| 0.0928 | 0.2414 | AI840335 | EST |
| 0.0926 | 0.1414 | AI841809 | SMT3 (supressor of mif two, 3) homolog 1 (S. cerevisiae) |
| 0.0846 | 0.0727 | AI840673 | ADP-ribosylation-like factor 6 interacting protein |
| 0.0747 | 0.0932 | AI836826 | glycoprotein 38 |
| 0.0702 | 0.1653 | AI842983 | EST |
| 0.0702 | 0.0298 | AI842681 | cartilage associated protein |
| 0.0684 | 0.2246 | AI844626 | RIKEN cDNA 1810003P21 gene |
| 0.0577 | 0.1983 | AI842554 | ESTs |
| 0.0533 | 0.1191 | AI841798 | tissue inhibitor of metalloproteinase 3 |
| 0.0495 | 0.0442 | AI836468 | myristoylated alanine rich protein kinase C substrate |
| 0.0492 | 0.2086 | AI839950 | four and a half LIM domains 1 |
| 0.0491 | 0.0287 | AI835976 | erythrocyte protein band 4.1-like 3 |
| 0.0477 | 0.1357 | AI840633 | carbohydrate (keratan sulfate Gal-6) sulfotransferase 1 |
| 0.0412 | 0.0659 | AI838614 | H19 fetal liver mRNA |
| 0.0364 | 0.2168 | AI835609 | neurofilament, light polypeptide |
| 0.0346 | 0.0267 | AI837752 | olfactomedin related ER localized protein |
| 0.0322 | 0.0796 | AI844038 | HGF-regulated tyrosine kinase substrate |
| 0.0284 | 0.0214 | AI842703 | procollagen, type III, alpha 1 |
| 0.0277 | 0.0498 | AI838607 | thrombospondin 1 |
| 0.0204 | 0.0109 | AI849859 | four and a half LIM domains 1 |

### Example 8

### Fluorescent Nucleotides

This example describes methods to prepare nucleotides containing at least one fluorophore; such nucleotides may be used as the modified nucleotide incorporated into modified random primers as disclosed herein. When a the modified nucleotide used to make such random primers comprises a fluorophore, it is not necessary to react the modified primers, or probes prepared using these primers, with a separate fluorophore (as described for some embodiments above).

In addition, this example lists some sources of commercially available fluorescent nucleotides that can be used in the present disclosure. Other commercial sources will be known to, or can be readily ascertained by, one of ordinary skill in the art.

NEN Life Science Products (Boston, Mass.) offers all four deoxynucleotides and ribonucleotide analogs with fluorophores attached. There are several different fluorophores available including fluorescein, Texas Red®, tetramethylrhodamine, coumarin, napthofluorescein, cyanine-3, cyanine-5, and Lissamine™. In addition, Molecular Probes (Eugene, Oreg.) sells deoxyuridinetriphosphate (dUTP) labeled with various fluorophores replacing the methyl group of thymine, synthesized by the method of U.S. Pat. No. 5,047,519. Because these nucleotides have 3′ hydroxyls, they can be used directly for synthesis reactions.

Alternatively, nucleotides containing other fluorophores can be prepared. The fluorophores are capable of being attached to the nucleotide, are stable against photobleaching, and have high quantum efficiency. In specific embodiments, the fluorophore does not interfere excessively with the degree or fidelity of nucleotide incorporation in the in vitro synthesis reaction used to produce the modified primers described herein. For instance, after attaching a fluorophore, the nucleotide is still able to undergo polymerization, complementary base pairing, and retains a free 3′ hydroxyl end.

The fluorophore can either be directly or indirectly attached to the nucleotide, though it is more commonly indirectly attached. For instance, the fluorophore may be attached indirectly to the nucleotide by a linker molecule. For example, a streptavidin linkage may be used.

Alternatively, the modified nucleotide to which the fluorophore is attached comprises, as part of the modification, a spacer (such as a carbon chain of about 2 to 15 atoms) that links the fluorophore (or reactive group with which the fluorophore reacts) to the nucleotide. U.S. Pat. Nos. 5,047,519 and 5,151,507 to Hobbs et al. (herein incorporated by reference) teach the use of linkers to separate a nucleotide from a fluorophore. Examples of linkers may include a straight or branched chain aliphatic group, particularly a alkyl group, such as C₁-C₂₀, optionally containing within the chain double bonds, triple bonds, aryl groups or heteroatoms such as N, O or S. Substituents on a diradical moiety can include C₁-C₆ alkyl, aryl, ester, ether, amine, amide or chloro groups.

### Example 9

### Other Uses for Modified Primer Labeling

Modified primers provided herein can be used in any method that requires nucleic acid labeling. The following are examples of known methods that incorporate the modified primers provided herein in order to generate a labeled product.

Use of Modified Primers in Dendrimer Labeling

In this example dendrimers, highly branched DNA molecules, are labeled using a modified primer as provided herein, for example an amine-modified primer. The modified primers contain a sequence in the 5′ end that is complementary to a sequence on a dendrimer arm, and that allows the primer to bind to the dendrimer. The 5′ end of the modified primer also contains a modified base, such as an amino allyl-modified base, to which label detection molecules can be added. Amine modified primers containing amine-modified nucleotides can be synthesized using in vitro chemical synthesis as is described herein. Examples of label detection molecules include, but are not limited to, fluorescent molecules and biotin. The labeled dendrimers are used, for instance, to hybridize to a cDNA probe. cDNA probes labeled in this manner can be used to generate hybridization signals, for instance in microarrays. The use of dendrimers, once they are labeled, is known (see, for example, products and procedures recommended by Genisphere, Hatfield, Pa.).

Indirect Labeling and Detection of cDNA Using Tyramide Signal Amplification (TSA)

Tyramide signal amplification (TSA) provides a consistent and reproducible signal amplification method for cDNA microarray analysis. Modified random hexamers, as described herein, for instance with fluorescein or biotin added at one end, can be used as primers to synthesize labeled cDNA probes from small amounts of total RNA. Purified fluorescein and biotin labeled cDNAs are hybridized to microarrays and the TSA detection method is applied as described in Karsten et al., Nucleic Acids Research, 30:E4, 2002.

Labeling RAA Fragments Generated by the DATAS Technique

Methods can be used to label RNA fragments generated by the DATAS technique, thereby allowing for a more accurate and sensitive comparative study of splicing events that characterize distinct physiopathological situations. RNA fragments generated by the DATAS technique (Schweighoffer et al., Pharmacogenomics, 1:187-197, 2000) can be reverse transcribed or amplified and labeled using amine-modified random primers that are synthesized as described herein.

### Example 11

### Kits for Labeling Probes and Assaying Arrays

The modified random primers disclosed herein can be supplied in the form of a kit for use in preparing labeled probes, for instance preparation of a hybridization probe suitable for assaying a microarray. In specific examples of such kits, an appropriate amount (e.g. sufficient to prime one or more labeling reactions) of modified random primers is provided in one or more containers. The primers may be provided suspended in an aqueous solution or as a freeze-dried or lyophilized powder, for instance. The container(s) in which the primers are supplied can be any conventional container that is capable of holding the supplied form, for instance, microfuge tubes, ampoules, or bottles. In some applications, primers may be provided in pre-measured single use amounts in individual, typically disposable, tubes or equivalent containers. With such an arrangement, the sample to be labeled can be added to the individual tubes and reactions carried out directly.

The amount of each primer supplied in the kit can be any appropriate amount, depending for instance on the market to which the product is directed For instance, if the kit is adapted for research or clinical use, the amount of each random primer provided would likely be an amount sufficient to label several hybridization probes. Those of ordinary skill in the art know the amount of primer that is appropriate for use in a single labeling reaction; specific examples disclosed herein provide additional guidance.

In some embodiments of the current invention, kits may also include the reagents necessary to carry out amplification, polymerization, transcription, or other reactions, including, for instance, DNA or RNA sample preparation reagents, appropriate buffers (e.g., transcription or polymerase buffer), salts (e.g., magnesium chloride), deoxyribonucleotides (dNTPs), and/or modified nucleotides (e.g., aa-dUTP).

Kits may additionally include one or more buffers for use during assay of an array. For instance, such buffers may include a low stringency wash, a high stringency wash, and/or a stripping solution.

Buffers or other constituents provided with kits herein may be provided in bulk, where each container of is large enough to hold sufficient reagent for several isolation, polymerization, probing, washing, or stripping procedures. Alternatively, the reagents can be provided in pre-measured aliquots, which might be tailored to the size and style of the kit.

Certain kits may also provide one or more containers in which to carry out array-probing reactions.

Kits may in addition include either labeled or unlabeled control probe molecules, to provide for internal tests of either the labeling procedure or probing of an array, or both. The control probe molecules may be provided suspended in an aqueous solution or as a freeze-dried or lyophilized powder, for instance. The container(s) in which the controls are supplied can be any conventional container that is capable of holding the supplied form, for instance, microfuge tubes, ampoules, or bottles. In some applications, control probes may be provided in pre-measured single use amounts in individual, typically disposable, tubes or equivalent containers.

The amount of each control probe supplied in the kit can be any particular amount, depending for instance on the market to which the product is directed. For instance, if the kit is adapted for research or clinical use, sufficient control probe(s) likely will be provided to perform several controlled analyses of the array. Likewise, where multiple control probes are provided in one kit, the specific probes provided will be tailored to the market and the accompanying kit.

This disclosure provides methods of producing modified nucleic acid molecules, including labeled nucleic acids, for use in hybridization reactions, using modified random primers to initiate synthesis. The disclosure further provides modified random primers, modified probe nucleic acid molecules produced by methods disclosed herein, and methods of using these molecules. It will be apparent that the precise details of the methods and compositions described may be varied or modified without departing from the spirit of the described invention. All such modifications and variations that fall within the scope and spirit of the claims below are claimed.

1. A method of producing a modified nucleic acid probe, comprising
contacting a nucleic acid template with a modified random primer under conditions sufficient to permit base-specific hybridization between the template and the primer, wherein the modified random oligonucleotide primer comprises an amine-modified dNTP or a label-substituted dNTP; and
polymerizing a nucleic acid molecule complementary to a nucleic acid sequence in the template and incorporating at least one modified oligonucleotide primer, thereby producing the modified nucleic acid probe.

2. The method of claim 1, wherein the modified random primer is modified at the five prime end of the primer.

3. The method of claim 1, wherein the modified random primer comprises an amine-modified dNTP, the method further comprising
coupling the modified nucleic acid probe to a label molecule to form a label-probe conjugate.

4. A modified random primer for use in the method of claim 1.

5. The modified primer of claim 4, wherein the primer is any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

6. The modified random primer of claim 4, wherein the primer is P2 (SEQ ID NO: 1).

7. The modified random primer of claim 4, wherein the primer is P4 (SEQ ID NO: 2).

8. The method of claim 1, wherein the nucleic acid template comprises a mixture of nucleic acid molecules.

9. The method of claim 8, wherein the mixture of nucleic acid molecules comprises RNA.

10. The method of claim 9, wherein polymerizing comprises polymerizing a cDNA.

11. A method of producing a fluorescent hybridization probe, comprising
contacting a template nucleic acid sample with a modified random primer comprising at least one aminoallyl dUTP residue;
polymerizing a nucleic acid molecule complementary to a sequence in the template sample and incorporating one or more modified random primers, to produce a modified complementary nucleotide; and
contacting the modified complementary nucleotide with an amine-reactive fluorescent label, thereby producing the fluorescent hybridization probe.

12. The method of claim 11, wherein aminoallyl dNTP is included during polymerizing.

13. The method of claim 11, wherein the template nucleic acid comprises mRNA and polymerizing comprises reverse transcription.

14. A fluorescent hybridization probe produced by the method of claim 11.

15. An improved method for random primer reverse transcription labeling of a nucleic acid hybridization probe, the improvement comprising using random primers modified with at least one amine-substituted dNTP or fluorescent-dye modified dNTP in the reverse transcription reaction.

16. An improved hybridization probe as produced by the method of claim 15.

17. The method of claim 1, wherein the nucleic acid template is an amplified nucleic acid template.

18. A kit for producing a labeled hybridization probe or for probing an array, comprising the modified random primer of claim 4.

19. The method of claim 1, wherein the nucleic acid template is originally isolated from a small number of cells.

20. The method of claim 19, wherein the small number of cells is lysed by sonication in a buffer comprising first strand buffer and an RNase inhibitor.

21. The method of claim 19, wherein the small number of cells is less than about 1000 cells.

22. The method of claim 19, wherein the small number of cells is less than about 100 cells.

23. The method of claim 19, wherein the small number of cells is about 10 cells.

24. The method of claim 19, wherein the small number of cells is about 1 cell.

25. The method of claim 19, wherein the nucleic acid template is an amplified template.

26. The method of claim 25, wherein the amplified template comprises RNA.

27. The method of claim 26, further comprising contacting the amplified template with a second primer, wherein the second primer has a nucleic acid sequence as set forth in SEQ ID NO: 12, under conditions sufficient to permit base-specific hybridization between the template and the second primer.

28. The method of claim 27, wherein the second primer, comprising a nucleic acid sequence as set forth in SEQ ID NO: 12, is used in at least one round of cDNA synthesis other than the first round.

29. The method of claim 27, wherein the modified random primer comprises an amine-modified dNTP, the method further comprises coupling the amine-modified nucleic acid probe to a label molecule to form a label-probe conjugate.

30. A method of producing an RNA template from a small number of cells, comprising:
lysing a small number of cells by sonication in a buffer, wherein the buffer comprises first strand buffer and an RNase inhibitor, to produce a lysate,
wherein the lysate comprises the RNA nucleic acid template.

31. The method of claim 30, wherein the small number of cells comprises less than about ten cells.

32. The method of claim 30, wherein the small number of cells comprises about one cell.

33. A method of producing a modified nucleic acid probe, comprising:
amplifying the RNA template of claim 30 to produce an amplified template;
generating cDNA from the amplified template;
contacting the cDNA with a modified random primer comprising an amine-modified dNTP under conditions sufficient to permit hybridization between the cDNA and the modified random primer; and
polymerizing a nucleic acid molecule complementary to a nucleic acid sequence in the cDNA and incorporating at least one modified oligonucleotide primer, thereby producing the modified nucleic acid probe.


SEQUENCE LISTING (13 sequences)

SEQ ID NO: 1 - DNA, 7 bp, Artificial sequence
  oligonucleotide sequence
nnnnnnn

SEQ ID NO: 2 - DNA, 6 bp, Artificial sequence
  oligonucleotide sequence
nnnnnn

SEQ ID NO: 3 - DNA, 8 bp, Artificial sequence
  oligonucleotide sequence
nnnnnnnn

SEQ ID NO: 4 - DNA, 9 bp, Artificial sequence
  oligonucleotide sequence
nnnnnnnnn

SEQ ID NO: 5 - DNA, 10 bp, Artificial sequence
  oligonucleotide sequence
nnnnnnnnnn

SEQ ID NO: 6 - DNA, 11 bp, Artificial sequence
  oligonucleotide sequence
nnnnnnnnnnn

SEQ ID NO: 7 - DNA, 11 bp, Artificial sequence
  oligonucleotide sequence
nnnnnnnnnnn

SEQ ID NO: 8 - DNA, 13 bp, Artificial sequence
  oligonucleotide sequence
nnnnnnnnnnnnn

SEQ ID NO: 9 - DNA, 14 bp, Artificial sequence
  oligonucleotide sequence
nnnnnnnnnnnnnn

SEQ ID NO: 10 - DNA, 7 bp, Artificial sequence
  oligonucleotide sequence
nnnnnnn

SEQ ID NO: 11 - DNA, 55 bp, Artificial sequence
  oligonucleotide sequence
aaacgacggccagtgaattgtaatacgactcactataggcgcttttttttttttt

SEQ ID NO: 12 - DNA, 41 bp, Artificial sequence
  oligonucleotide sequence
gcgcgaaattaaccctcactaaagggagagggnnnnnnnnn

SEQ ID NO: 13 - DNA, 59 bp, Artificial sequence
  oligonucleotide sequence
ggccagtgaattgtaatacgactcactatagggaggcggtttttttttttttttttttt